(19) United States
(12) Patent Application Publication
(10) Pub. No.: US 2026/0193337 A1
(43) Pub. Date: Jul. 9, 2026

(54) METHOD FOR MAINTAINING QUIESCENCE OF EARLY STAGE DUPUYTREN'S DISEASE IN INDIVIDUALS ACHIEVING QUIESCENCE AFTER RECEIVING FOUR INJECTIONS OF ADALIMUMAB OVER TWELVE MONTHS

(71) Applicant: Oxford University Innovation Limited, Oxford (GB)

(72) Inventors: Jagdeep Nanchahal, Headington (GB); Helen A. Dakin, Bicester (GB)

(73) Assignee: Oxford University Innovation Limited, Oxford (GB)

(21) Appl. No.: 19/125,546

(22) PCT Filed: Nov. 2, 2023
(86) PCT No.: PCT/IB2023/061090

§ 371 Date: Apr. 29, 2025

Related U.S. Application Data
(60) Provisional application No. 63/382,196, filed on Nov. 3, 2022.

(51) Int. Cl. C07K 16/24; A61K 39/00; A61P 19/04

(52) U.S. Cl. CPC C07K 16/241; A61P 19/04; A61K 2039/54; A61K 2039/545; C07K 2317/21; C07K 2317/76

(57) ABSTRACT

This invention provides a method for maintaining quiescence in an early stage Dupuytren's disease nodule on the hand of an individual who has achieved quiescence in such nodule after receiving four initial injections of a liquid pharmaceutical composition containing 40 mg of adalimumab into such nodule.

This application is a § 371 national stage of PCT International Application No. PCT/IB2023/061090, filed Nov. 2, 2023, claiming the benefit of U.S. Provisional Application No. 63/382,196, filed Nov. 3, 2022, the contents of each of which are hereby incorporated by reference into the subject application.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### TECHNICAL FIELD

This disclosure relates to a method for maintaining quiescence in an early stage Dupuytren's disease nodule on the hand of an individual who has achieved quiescence in such nodule after receiving four initial injections of a liquid pharmaceutical composition containing 40 mg of adalimumab into such nodule.

### BACKGROUND OF THE INVENTION

Dupuytren's disease (DD) is a common fibrotic hand condition affecting 12% of 55-year-olds and 29% of 75-year-olds in Western populations.¹ Early-stage DD presents as nodules on the palmar aspect of the hand that progress to form cords. The cords of late-stage DD cause curling of the finger joints (flexion deformity), impairing hand function and quality of life.² Treatment options for late-stage DD include surgical excision, needle fasciotomy and collagenase injections.³ However, all have limitations, including risk of recurrence.⁴ Although several interventions (e.g. steroid injections or radiotherapy), are used to treat early-stage DD, none have been compared against other interventions in randomized controlled trials (RCTs).⁵

### SUMMARY OF THE INVENTION

This invention provides a method for maintaining quiescence in an early stage Dupuytren's disease nodule on the hand of an individual who has achieved quiescence in such nodule after receiving four initial injections of a liquid pharmaceutical composition containing 40 mg of adalimumab into such nodule, which comprises:
injecting the same pharmaceutical composition into the same nodule four times at a time interval of three months between each injection over a time period of 12 months commencing at least 18 months after the fourth of the initial four injections, wherein
(A) quiescence in such nodule is defined as meeting the following three criteria 18 months after the first of the initial four injections:
   (1) decrease or no change in nodule area,
   (2) decrease or no increase in nodule hardness, and
   (3) less than or equal to a 5 degree increase in active flexion deformity,
(B) the initial four injections are administered at a time interval of three months between each injection over a time period of 12 months using a syringe with a 25-gauge needle, and
(C) the pharmaceutical composition is citrate-free and has a volume of 0.4 ml.

### BRIEF DESCRIPTION OF THE DRAWINGS

FIG. 1: Model structure. The model simulates disease history for individual RIDD participants at discrete six-month time intervals. Eighteen months after initial treatment, individual patients either continue to have progressive early-stage DD, enter quiescent early-stage DD or progress to late-stage DD; the probability of each outcome varies between treatments. Patients with late-stage DD progress directly to the untreated late-stage DD state. Quiescent patients have no change in flexion deformity for a certain number of years (Box 1). After that period, they will either be retreated or have progressive early-stage DD (depending on treatment group). Patients with progressive early-stage DD experience progressive changes in flexion deformity (although these are shown as linear in the figure, in practice the change in flexion deformity each six-month period varies stochastically and may be negative). When the flexion deformity exceeds 30°, patients enter the untreated late-stage DD state. In late-stage DD, patients may undergo a pre-specified series of surgical procedures. In the base case analysis, this comprised up to three percutaneous needle fasciotomies (PNF), no more than one limited fasciectomy and no more than one dermofasciectomy. Patients who failed any PNF procedure and opted for further surgery received limited fasciectomy rather than PNF. Transitions in late-stage DD were modelled using the Markov disease states used by Brazzelli et al,⁴ although our model captures longer sequences of surgical interventions. The model also allows for death from any disease stage.

FIG. 2: Cost-effectiveness acceptability curves showing the probability that each treatment strategy is best value for money at different values that the NHS may be willing or able to pay to gain one quality-adjusted life-year (QALY). For example, if the NHS were willing to pay £30,000 per QALY gained, there is a 93% probability that repeated adalimumab is best value for money out of the three treatments shown, a 7% probability that standard care is best and <1% probability that one course of adalimumab is best value for money. The frontier shows the probability of being cost-effective for the treatment that is expected to be best value for money at each ceiling ratio; the difference between the frontier and 1 indicates the decision uncertainty: i.e. the probability of adopting a treatment that is not in fact best value for money.

FIG. 3-eFIG. 1: Cost-effectiveness acceptability curve (12-month follow-up)

FIGS. 4A and 4B-eFIG. 2: Tornado diagram showing the results of (FIG. 4A) one-way sensitivity analyses and (FIG. 4B) scenario analyses on the cost per quality-adjusted life-year (QALY) for repeated courses of adalimumab compared with standard care. In panel (A) the 20 parameters that had the greatest effect on the results are shown in descending order; the black bars represent the cost/QALY using the maximum value for each parameter, while the white bars represent the cost/QALY using the minimum value. All other parameters were held at their point estimates; since the model is non-linear, base case results therefore differ slightly from the average across probabilistic runs shown in Table 2. The ranges over which each variable was varied in one-way sensitivity analyses are given in eTable 11.

FIG. 5-eFIG. 3: Scatter graph on cost-effectiveness plane: 1 course adalimumab vs. standard care

FIG. 6-eFIG. 4: Scatter graph on cost-effectiveness plane: repeated adalimumab vs. standard care

FIG. 7-eFIG. 5: expected value of perfect information (EVPI) per patient. Using the Sheffield Accelerated Value of Information,³⁸ most influential individual parameter was the utility of quiescence, followed by the probability that adalimumab-treated patients have late-stage DD at 18 months. The EVPI for the group of parameters estimated from the RIDD sample was £97.96 per patient (£272 million for the population); this could be viewed as the maximum benefit from a confirmatory phase III trial similar to RIDD. Collecting additional data, or extended follow-up, in order to also get data on the duration of quiescence increased the maximum value of such a trial by £7.48 per patient (£19 million for the population) to £105. Eliminating all uncertainty around the parameters relating to the costs, utilities, success rates, recurrence rates and uptake of interventions for late-stage DD would be valued at £79 per patient (£205 million for the population) for the decision about adalimumab treatment alone, excluding the benefits that such information would have for informing decisions about the best course of treatment once patients develop late-stage DD. Scenario analysis: additional comparators: Adding radiotherapy as a 4^{th} treatment option within the base case analysis increased EVPI by only £21 to £293 per patient at a £20,000/QALY ceiling ratio. The probability radiotherapy being the best value for money out of the treatments considered never exceeded 0.5% (figures not shown because they are in distinguish from the base case). Adding steroids as a fourth option within the base case analysis using the steroid scenario B decreased the EVPI to £80 per patient at a £20,000/QALY ceiling ratio.

FIG. 8-eFIG. 6: Cost-effectiveness acceptability curve for steroid scenario B, adding steroids as a 4^{th} treatment option

FIG. 9-eFIG. 7: EVPI for steroid scenario B, adding steroids as a 4^{th} treatment option

### DETAILED DESCRIPTION

### Embodiments of the Invention

The following embodiments and examples (including details thereof) are set forth to aid in an understanding of the subject matter of this disclosure but are not intended to, and should not be construed to, limit in any way the invention that is claimed.

This invention provides a method for maintaining quiescence in an early stage Dupuytren's disease nodule on the hand of an individual who has achieved quiescence in such nodule after receiving four initial injections of a liquid pharmaceutical composition containing 40 mg of adalimumab into such nodule, which comprises:
injecting the same pharmaceutical composition into the same nodule four times at a time interval of three months between each injection over a time period of 12 months commencing at least 18 months after the fourth of the initial four injections, wherein
(A) quiescence in such nodule is defined as meeting the following three criteria 18 months after the first of the initial four injections:
   (1) decrease or no change in nodule area,
   (2) decrease or no increase in nodule hardness, and
   (3) less than or equal to a 5 degree increase in active flexion deformity,
(B) the initial four injections are administered at a time interval of three months between each injection over a time period of 12 months using a syringe with a 25-gauge needle, and
(C) the pharmaceutical composition is citrate-free and has a volume of 0.4 ml.

In some embodiments, the injecting commences 18 to 24 months after the fourth of the initial four injections.

In some embodiments, the injecting commences 24 to 36 months after the fourth of the initial four injections.

In some embodiments, the injecting commences 36 to 48 months after the fourth of the initial four injections.

In some embodiments, the injecting commences two to five years after the fourth of the initial four injections.

In some embodiments, the injecting commences five to ten years after the fourth of the initial four injections.

### Experimental Details

### Example 1: Cost-Effectiveness of Adalimumab for Early-Stage Dupuytren's Disease: An Economic Evaluation Based on a Randomized Controlled Trial and Individual-Patient Simulation Model

### Overview

Aims: To estimate the potential cost-effectiveness of adalimumab compared with standard care alone for the treatment of early-stage Dupuytren's disease and the value of further research from a NHS perspective.

Patients: Data from the RIDD randomized controlled trial of intranodular adalimumab injections in patients with early-stage progressive Dupuytren's disease was used. RIDD found that intranodular adalimumab injections reduced nodule hardness and size in patients with early-stage Dupuytren's disease, indicating the potential to control disease progression.

Methods: A within-trial cost-utility analysis compared four adalimumab injections with no further treatment against standard care alone, taking a 12-month time horizon and using prospective data on EQ-5D-5L and resource use from the RIDD trial. We also developed a patient-level simulation model similar to a Markov model to extrapolate trial outcomes over a lifetime using data from the RIDD trial and a literature review. This also evaluated repeated courses of adalimumab each time the nodule reactivated (every three years) in patients who initially responded.

Results: The within-trial economic evaluation found that adalimumab plus standard care cost £503,410 per quality-adjusted life-year (QALY) gained vs. standard care alone over a 12-month time horizon. The model-based extrapolation suggested that, over a lifetime, repeated courses of adalimumab could cost £14,593 (95% confidence interval: £7,534, £42,698) per QALY gained vs. standard care alone. If the NHS were willing to pay £20,000/QALY gained, there is a 77% probability that adalimumab with retreatment is best value for money.

Conclusion: Repeated courses of adalimumab are likely to be a cost-effective treatment for progressive early-stage Dupuytren's disease. The value of perfect parameter information that would eliminate all uncertainty around the parameters estimated in RIDD and the duration of quiescence was estimated to be £105 per patient or £272 million for all 2,584,411 prevalent cases in the UK.

### INTRODUCTION

Dupuytren's disease (DD) is a common fibrotic hand condition affecting 12% of 55-year-olds and 29% of 75-year-olds in Western populations.¹ Early-stage DD presents as nodules on the palmar aspect of the hand that progress to form cords. The cords of late-stage DD cause curling of the finger joints (flexion deformity), impairing hand function and quality of life.² Treatment options for late-stage DD include surgical excision, needle fasciotomy and collagenase injections.³ However, all have limitations, including risk of recurrence.⁴ Although several interventions (e.g. steroid injections or radiotherapy), are used to treat early-stage DD, none have been compared against other interventions in randomized controlled trials (RCTs).⁵

Tumour necrosis factor (TNF) was identified as a potential therapeutic target,^{6,7} and the RIDD phase 2a dose-ranging trial found that injecting adalimumab directly into DD nodules downregulated myofibroblasts,⁸ the effector cells in DD. The RIDD phase 2b RCT found that adalimumab injections resulted in softening and reduction in size of early-stage progressive DD nodules, which continued to decrease further for nine months after the final injection.⁹ To date, RIDD is the only trial evaluating adalimumab in DD.

It is important to evaluate the extent to which the cost of early-stage DD treatment is offset by reductions in subsequent surgery and whether quality of life improvements are worth the additional cost. No previous studies have evaluated the cost-effectiveness of treatments for early-stage DD.¹° Early economic evaluations are often used to identify whether treatments that are not yet licensed could be cost-effective and identify where future research would be most valuable.¹¹

This study aimed to use RIDD trial data¹² to assess the cost-effectiveness of adalimumab vs. standard care in the UK NHS setting. The within-trial analysis compared costs and quality of life over one year. To determine longer-term outcomes, we built a patient-level simulation model to assess whether adalimumab (with/without retreatment) has the potential to be cost-effective for progressive early-stage DD, and estimate the value of further research.

### Methods

Full details of the RIDD (ISRCTN27786905, ClinicalTrials.gov NCT03180957) trial protocol¹³ and health economics analysis plan (HEAP) 12 have been published. For both the within-trial and model-based economic evaluation, the patient population comprised adults with progressive early-stage DD who met the RIDD inclusion criteria: active extensor deficit ≤30° and an established, clinically-distinct nodule with a clear history of progression in the preceding six months.⁹

Both analyses took a National Health Service (NHS) and personal and social services (PSS) perspective, following the NICE reference case.¹⁴ Following UK guidelines,¹⁴ we conducted cost-utility analysis, measuring health outcomes in quality-adjusted life-years (QALYs). The economic evaluation compared adalimumab plus standard care against standard care alone, as most early-stage DD patients are currently not treated. The standard care group received placebo (saline) injection within the trial. The index year for costs was 2018-9. Each course of adalimumab comprised four doses of 40 mg adalimumab in 0.4 mL (Humira, AbbVie Ltd, Maidenhead UK) administered at three-month intervals by a consultant in an outpatient setting, with the option of topical local anaesthetic cream. Each dose, including administration, cost £475 (eTable 1).

### Within-Trial Economic Evaluation

Here the cost-effectiveness of adalimumab vs. standard care was assessed using quality-adjusted life-years (QALYs) as the main outcome, assessed by the EQ-5D-5L questionnaire at baseline, 3, 6, 9, 12 and 18 months. Resource use related to DD in the injected digit was assessed at the same timepoints, including injections and surgery performed and self-reported health and social care usage. An intention-to-treat cost-utility analysis was performed using data collected up to 12 months post-randomization; patients were analysed on an as-randomized basis, regardless of adherence to the protocol. Missing data were handled using multiple imputation. No discounting was used as the time horizon of primary interest was 12 months. We estimated the incremental cost-effectiveness ratio by dividing the mean cost difference between adalimumab and standard care by the mean QALY difference. The joint uncertainty around these estimates was explored using bootstrapping. We used sensitivity analyses to assess the impact of different drug costs, and adherence to the protocol. eMethods 1 provides additional details on unit costs and methodology for the within-trial analysis.

### Patient-Level Simulation Model

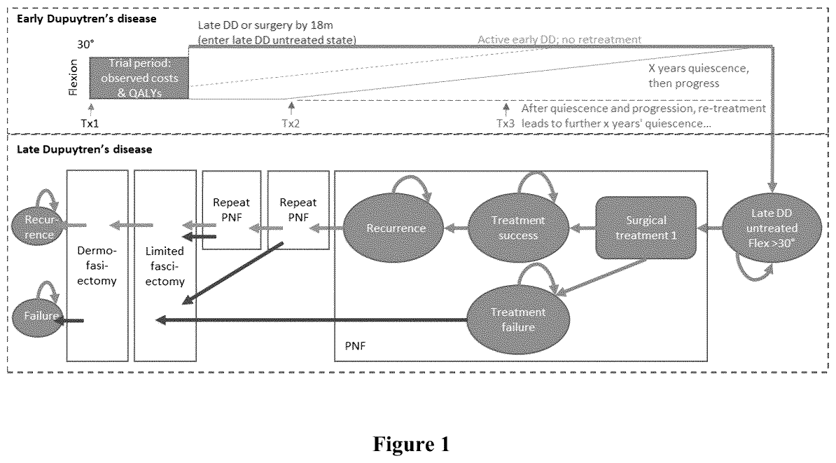

An individual-patient simulation model with a structure comparable to a Markov model was used to extrapolate individual-patient data from the RIDD trial. This provides early modelling of the lifetime cost-effectiveness of adalimumab and the value of further research. The structure of the model (FIG. 1) was based on the conceptual model pre-specified within the HEAP,¹² which was informed by a systematic review of previous economic evaluations.¹⁰ eMethods 2 gives further details on the model, data inputs and assumptions.

Individual-patient data was extrapolated for 69 UK RIDD participants randomized to placebo with complete baseline data. The model simulated changes in flexion deformity and EQ-5D utility of each individual over time, depending on treatments and disease progression. Each individual was simulated 100 times for each of 1000 sets of parameter values under each treatment strategy to obtain stable estimates and minimize Monte Carlo error (eMethods 2). The model simulated disease progression and treatment of a single DD nodule from the end of the 18-month trial until death.

Nodule quiescence was used as an intermediate endpoint linking trial outcomes with progression to late-stage DD. Quiescence was defined as having all three of the following between baseline and 18 months: decrease or no change in nodule area; decrease or no change in nodule hardness; and ≤5° increase in active flexion deformity. Box 1 summarizes key model assumptions.

The model compared three treatment strategies:

Standard care: no treatment for early-stage DD. A proportion of patients will be quiescent for three years, after which flexion deformity will gradually increase.

One course of adalimumab: patients have four adalimumab injections and receive no further treatment for early-stage DD. A proportion of patients will be quiescent for three years, after which flexion deformity will gradually increase.

Repeated courses of adalimumab injections: patients who were quiescent after the initial four injections will maintain quiescence indefinitely by receiving further courses of four injections each time the nodule reactivates. Patients who were not initially quiescent will receive no further treatment for early-stage DD.

RIDD data were used to calculate the probability of achieving quiescence or developing late-stage DD by 18 months, the effect of quiescence on quality of life and the rate at which flexion deformity changes without treatment (eMethods 2). Data sources for model inputs that could not be reliably obtained from the RIDD sample were identified from a systematic review,¹⁰ supplemented, where necessary, by more recent data.¹⁵⁻¹⁷

Probabilistic sensitivity analysis quantified uncertainty, generating cost-effectiveness acceptability curves and estimates of the expected value of perfect information using published software¹⁸ (eMethods 2). All uncertain parameters were varied in one-way sensitivity analysis across their 95% confidence interval or the range reported in literature. Thirteen sensitivity analyses were also conducted (eMethods 2). When assessing cost-effectiveness, we assumed that the NHS would be willing or able to pay up to £20,000 per QALY gained.¹⁴

There is no approved treatment for early-stage DD, although treatments such as intranodular steroid injections or radiotherapy are used despite the lack of comparative RCT evidence.⁵ The studies to date are poor quality, nonrandomized, unblinded and do not report objective measures of nodule size, hardness or flexion deformity.⁵ The National Institute of Health and Care Excellence (NICE) found insufficient evidence to recommend radiotherapy outside research setttings.¹⁹ Although there was insufficient evidence to robustly assess the cost-effectiveness of adalimumab compared with other active treatments, we conducted scenario analyses comparing with radiotherapy and steroid injections (eMethods 2). Collagenase has been evaluated in a small trial for early-stage DD,²⁰ but was not considered here as it is only approved for late-stage disease and has previously been shown to not be cost-effective.^{4,15}

### Results

### Within-Trial Economic Evaluation

Two UK centres recruited 140 participants February 2017-April 2019.9 At 12 months, 91% of participants had quality of life data and 93% healthcare resource use (eTable 2).

EQ-5D-5L utilities remained relatively constant during the trial, with a mean between-group difference of 0.011 (95% confidence interval [CI]: −0.026, 0.048) at 12 months (eTable 3). Resource use among the trial population was low, with 2% (3/130) attending GP consultations, 2% (2/130) undergoing surgery and no patients reporting NHS physio/hand therapy in months 9-12 (eTable 5). Use of non-NHS resources was negligible (eTable 10). Total QALYs and NHS/PSS costs other than adalimumab did not differ significantly between randomized groups over the 12-month follow-up (p≥0.65; Table 1, eTable 7).

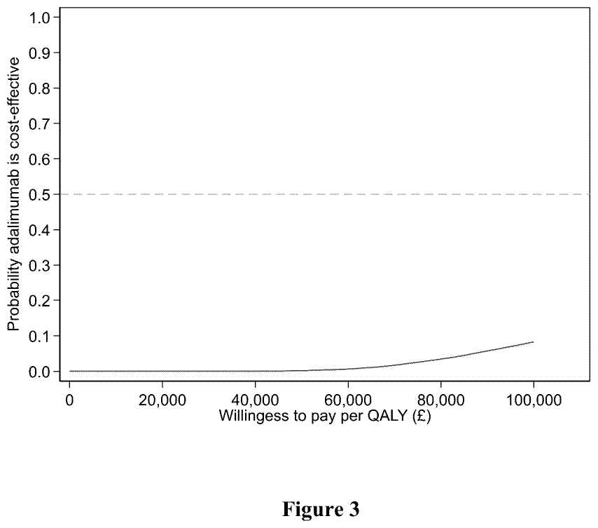

At a 12-month time horizon, adalimumab cost £503,410 per QALY gained and the probability of adalimumab injections being cost-effective at a willingness to pay threshold of £20,000/QALY was <1% (Table 1, eFIG. 1 (FIG. 3)). Our sensitivity analyses confirmed the base case finding that adalimumab was unlikely to be cost-effective over a 12-18 month time horizon at any realistic cost for the adalimumab injections (eTables 8-9).

The analyses to estimate model inputs (Table 2) showed that 21% of patients randomized to placebo had late-stage DD by 18 months and 22% were quiescent. Adalimumab-treated patients were 79% (95% CI: −4%, 211%; p=0.076) more likely to have quiescence at 18 months and marginally less likely to have late-stage DD or undergo surgery by 18 months (p=0.534). Patients meeting the criteria for quiescence at 18 months accrued 0.0395 (95% CI: 0.0079, 0.0704; p=0.018) more QALYs between 6 and 18 months than patients who did not have quiescence.

### Patient-Level Simulation Model

The simulation model suggested that over a lifetime, a single course of adalimumab delayed onset of late-stage DD by 0.31 (95% CI: −0.51, 1.31) years compared with standard care. Further courses of four adalimumab injections each time quiescence ended (in patients who were initially quiescent), gained an average of 1.81 (95% CI: 0.51, 3.10) years free of late-stage DD by preventing disease progression in 37% of patients. Consequently, patients who received a single course of adalimumab required 0.06 (95% CI: −0.10, 0.23) fewer operations for late-stage DD over a lifetime, compared with those who received standard care alone for early-stage DD (Table 3). Repeated adalimumab injections led to an average of 3.65 (95% CI: 2.35, 5.74) courses of four adalimumab injections and 0.44 (95% CI: 0.16, 0.73) fewer operations for late-stage DD compared with standard care.

One course of adalimumab gained 0.048 (95% CI: −0.053, 0.166) QALYs and cost an additional £1,746 (95% CI: £1,418, £2,060) compared with standard care. Repeated courses of adalimumab gained an average of 0.307 (95% CI: 0.110, 0.524) QALYs and cost an additional £4,484/patient (95% CI: £2,969, £6,859). Repeated courses of adalimumab cost £14,593 (95% CI: £7,534, £42,698) per QALY gained compared with standard care. A combination of standard care and repeated courses of adalimumab showed extended dominance over one course of adalimumab, being less costly and generating more QALYs.

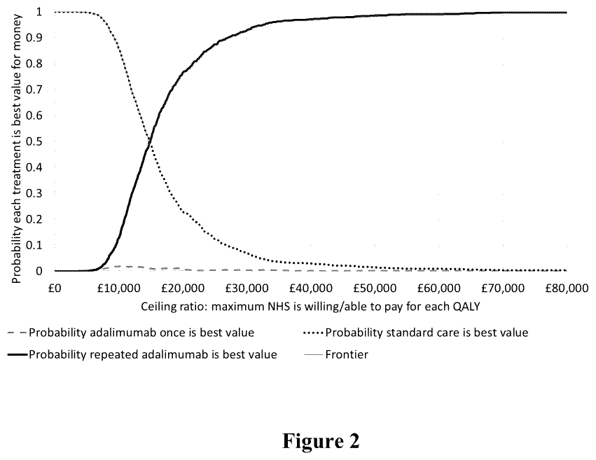

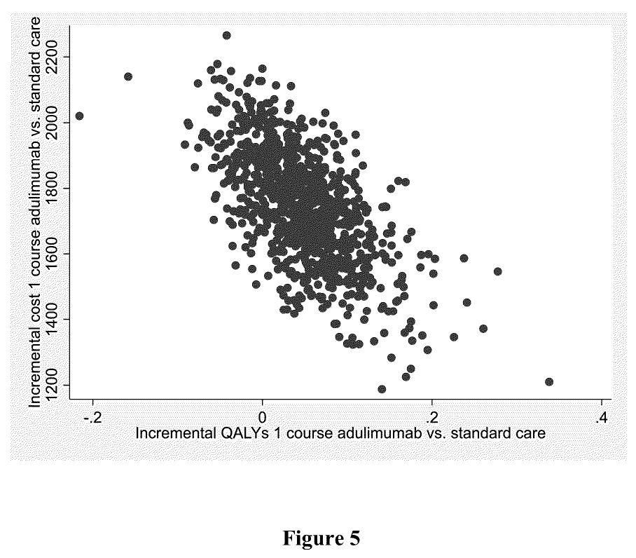

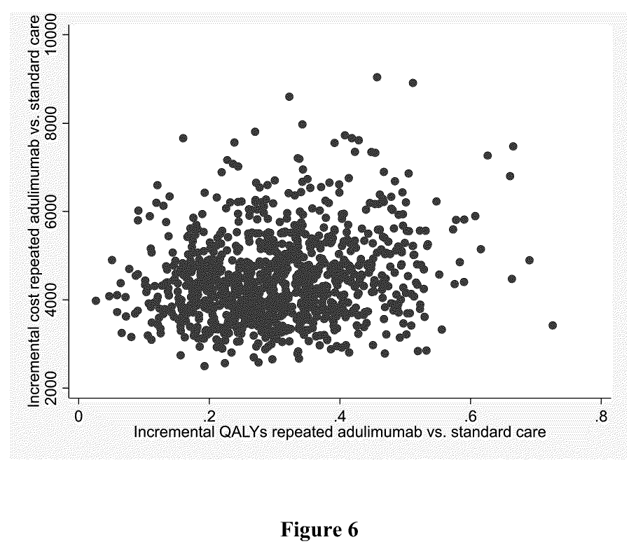

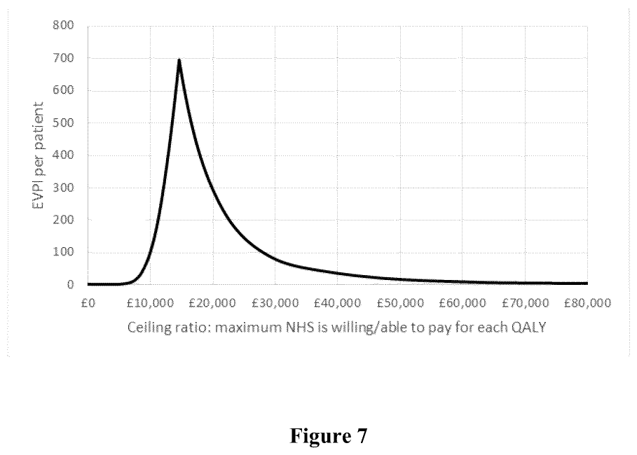

Probabilistic sensitivity analysis showed that if the NHS is willing to pay £20,000 per QALY gained,¹⁴ there is a 77% chance that repeated adalimumab courses are best value for money; this increased to 93% at a £30,000/QALY ceiling ratio (FIG. 2, eFIGS. 3-4 (FIGS. 5-6)). The expected value of perfect information that eliminates all uncertainty around the decision between the three treatment strategies was £292 per patient or £755 million for all 2,584,411 prevalent cases in the UK (eTable 12, eFIG. 5 (FIG. 7)). This represents the maximum amount that it would be worth spending on future research in this area. The maximum value of a confirmatory trial that eliminated uncertainty around the parameters estimated from RIDD data, plus the duration of quiescence was £105 per patient or £272 million for the UK population.

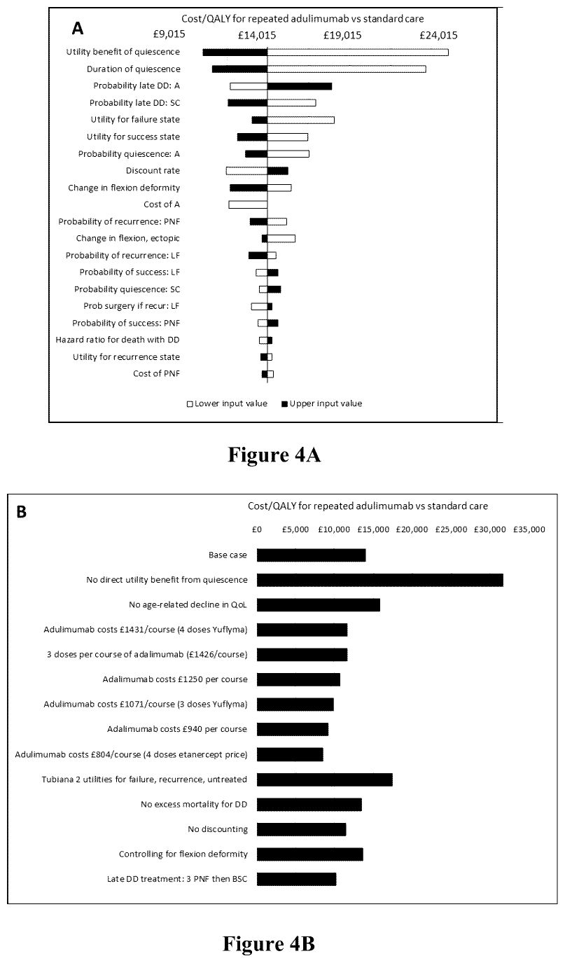

One-way sensitivity analyses (eFIG. 2 (FIGS. 4A and 4B)) demonstrated that changes in two model inputs could increase the cost per QALY gained for repeated adalimumab compared with standard care to >£20,000/QALY gained: reducing the utility gain associated quiescence to its lower 95% CI (0.0079), or reducing the duration of quiescence to 18 months. However, no single parameter change increased the cost-effectiveness ratio above £25,000/QALY and lower prices could reduce it.

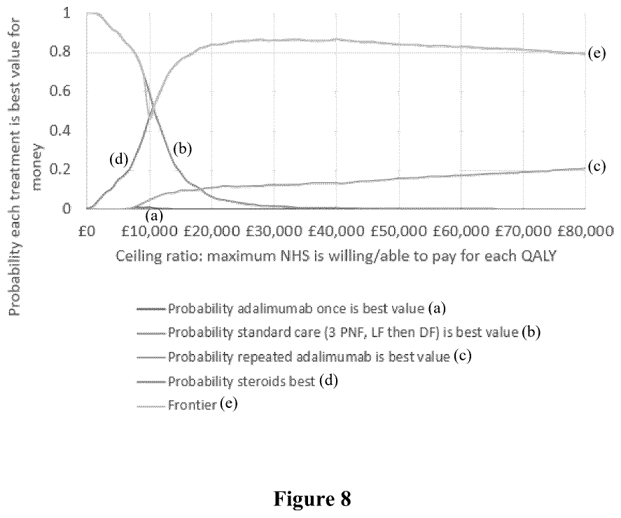

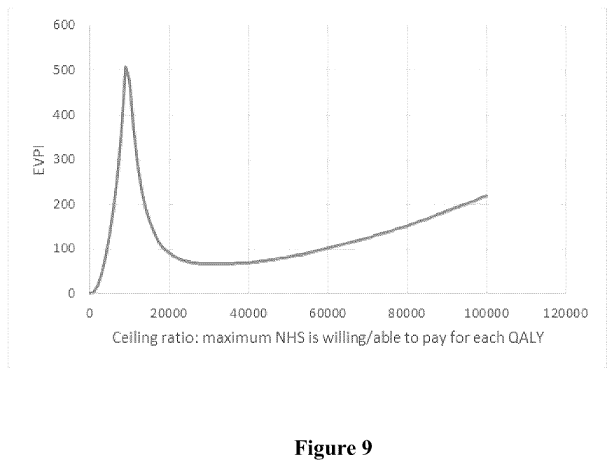

We also compared adalimumab and standard care against a single course of radiotherapy and against steroid injections every six months in quiescent patients, using additional assumptions due to the limited data. This demonstrated that radiotherapy would be strongly dominated by adalimumab and have <1% chance of being best value for money if it had the same efficacy as adalimumab, since it is more costly than one course of adalimumab (eFIGS. 6-7 (FIGS. 8-9), eTable 13). At £341/course, giving three steroid injections to quiescent patients every six months would be less costly than giving adalimumab every three years if steroids were at least as effective.

### DISCUSSION

There is currently no approved treatment for early-stage DD and patients are currently advised to wait until they have progressed and have flexion deformities limiting hand function before being offered surgery. The RIDD trial showed that a course of four adalimumab injections decreased nodule hardness and size.⁹ Here we assessed the cost-effectiveness of adalimumab for early-stage DD. Due to the low healthcare resource use, a difference in QALYs below the minimally-important difference for EQ-5D²¹ and small numbers of patients progressing to surgery (10 standard care group, 3 adalimumab group⁹), adalimumab was not cost-effective over a 12-18-month time horizon. However, DD is a slowly-progressive disease and the RIDD trial found that nodules continued to soften and reduce in size nine months after the final injection.⁹ We would expect most of the health gains from treatment to be experienced >12 months after start of treatment if those patients with reductions in nodule hardness and size are less likely to progress to late-stage DD and surgery. Therefore, we also performed long-term modelling.

The model showed that if adalimumab-induced quiescence lasted for three years, maintaining quiescence by further courses of four injections every three years would cost £14,593 per QALY gained compared with standard care. As treatments costing <£20,000/QALY are generally considered cost-effective,¹⁴ we can be 77% confident that repeated adalimumab is cost-effective compared with standard care (FIG. 2). The cost-effectiveness ratio would fall to £8,508/QALY if the price were similar to etanercept (eFIG. 2 (FIGS. 4A and 4B). Adalimumab prices vary substantially between countries, up to $3,380 (£2824) per dose in the USA,²² although this may change with the introduction of biosimilars in 2023. Whilst the duration of quiescence is unknown, nodules continued to soften and shrink up to the 18-month trial follow-up; 9 sensitivity analyses suggested that adalimumab would cost <£25,000/QALY gained even if quiescence lasted only 18 months.

RIDD is the largest randomized trial on early-stage DD⁴ and the first with prospective collection of costs and utilities. Key strengths of the within-trial economic evaluation include our comprehensive assessment of health and social services use and the robust estimation of unit costs. We achieved high levels of data completion, which reassures us that our results are both robust and representative of the trial population over the follow-up. Although there were more missing data at 18 months (partly attributable to the COVID-19 pandemic), we used multiple imputation to minimize the risk of bias. The COVID-19 pandemic may have affected quality of life and healthcare use after the 12-month follow-up. However, the observed data reflect the low healthcare use expected for early-stage DD, and we believe that the between-group comparisons presented are representative of our study population.

Only five previous model-based economic evaluations on DD have been published^{10,15} and this is the first to model early-stage progressive DD. Our model allows for longer sequences of treatments than previous studies. We found that a strategy of up to three percutaneous needle fasciotomies, followed by limited fasciectomy, then dermofasciectomy was cost-effective (£1,435/QALY gained) compared with providing only best supportive care after percutaneous needle fasciotomy (Appendix 3).

RIDD evaluated surrogate outcome measures (nodule hardness and size) over 18 months. Increased nodule size has been shown to correlate with development of late-stage finger contractures²³ and nodule hardness has been used in other early-stage DD studies.^{20,24,25} While ~10 years' follow-up would be required to provide level 1 evidence linking quiescence at 18 months with development of flexion deformities, we observed higher EQ-5D utility in patients achieving quiescence (Table 2) and more placebo-treated participants underwent/were awaiting surgery.⁹ Furthermore, when the nodules affected the proximal interphalangeal joint, placebo group participants developed greater flexion deformity.⁹ Taken together with evidence that anti-TNF down-regulates myofibroblast activity in vitro^{6,7} and in vivo,⁸ our data suggest that intranodular injections of adalimumab are likely to control disease progression.⁹ Further follow-up would be needed to test the assumption that patients who are quiescent after the first course of treatment will remain quiescent following retreatment. Further research on the natural history of early-stage DD would also inform economic evaluations for other interventions. The analysis also focused on treatment and progression of one nodule, but in practice patients may have many nodules at different stages and surgery for late-stage DD may treat several nodules simultaneously. Although it relies on assumptions, our model indicates that adalimumab is likely to be cost-effective and highlights the areas where further research is likely to be most valuable. The introduction of an effective treatment for early-stage DD may increase referrals; this was not factored in the analysis since the magnitude is unknown.

There are no RCTs on intralesional steroid injections for early-stage DD and the only RCT on radiotherapy was unblinded and compared two radiotherapy doses.^{5,9,26} NICE recommend that radiotherapy should not be used for DD outside research settings.¹⁹ Our results comparing adalimumab against these treatments should therefore be interpreted with caution and are intended only to give an indication of the spread of possible results and the value of further research.¹¹ The analysis excluded complications from steroids and radiotherapy.^{4,26} However, our study suggests that there would be little value in further research comparing radiotherapy vs. adalimumab.

These results suggest that adalimumab is likely to be a cost-effective treatment for progressive early-stage DD and that additional research is likely to be good value for money. The value of eliminating all uncertainty around this decision was found to be £755 million in improvements in health of UK patients and NHS savings. Adalimumab has the potential to transform management of DD.

### Take Home Message

In the trial-based economic evaluation, adalimumab cost £503,410 per quality-adjusted life-year (QALY) gained compared with standard care over 12 months.

The model-based extrapolation showed that over a lifetime, repeated courses of adalimumab are likely to cost £14,593 per QALY gained.

Repeated courses of adalimumab are likely to be a cost-effective way to treat early-stage Dupuytren's disease, compared with standard care.

Box 1: Key assumptions within the model. A full
list of assumptions is given in eMethods 2.

Six-month cycles.
55 year time horizon from randomisation (equal to the life expectancy²⁷ of the
youngest UK patient randomized to placebo in RIDD).
Costs and utilities beyond the 18-month trial were discounted at 3.5% per annum.¹⁴
Late-stage DD was defined as flexion deformity >30°. The transition to late-stage
DD was assumed to be permanent and patients cannot return to early-stage disease.
Quiescent patients were assumed to have 0% chance of progressing to late-stage
DD until quiescence ends. In the absence of external data on the duration of
quiescence, the base case analysis assumed that quiescence lasts for double the
duration of the trial (three years) regardless of whether quiescence was achieved
with adalimumab treatment or without treatment. This was varied between the trial
duration (1.5 years) and 5 years (~3 times the trial duration) in one-way and
probabilistic sensitivity analyses. The RIDD trial observed that adalimumab-treated
nodules continued to soften and reduce in size over the nine-month period between
end of treatment and 18 months,⁹ suggesting that the duration of quiescence is
likely to be substantially longer than 1.5 years. Natural history studies have shown
that many patients with untreated early-stage DD have periods of quiescence
(during which the nodule is inactive and cords are unlikely to develop) and periods
of activity (during which the nodule is more active and a cord may develop).²³ The
association between surface area and Tubiana stage (a categorical measure based on
flexion deformity)²³ supports the assumptions within the model regarding
quiescence and the link between quiescence, the definition of which includes
nodule size, and progression to flexion deformity.
As per the HEAP,¹² we assumed that patients successfully treated for early-stage
DD (defined here as quiescence) would not be at risk of disease progression for the
duration of that quiescence.
Patients in the adalimumab retreatment group were assumed to seek treatment as
soon as they noticed an increase in nodule size, pain, tenderness, or itching and
those patients who were quiescent after the initial adalimumab treatment were
assumed to maintain quiescence after retreatment; consequently, quiescence was
assumed to continue indefinitely. Within RIDD, there was no relationship between
the development of antibodies against adalimumab and response to treatment.⁹
Patients who did not reach quiescence with initial treatment were assumed to
receive no further treatment for early-stage DD.
Patients who develop late-stage DD were assumed to receive up to three
percutaneous needle fasciotomies (PNF), no more than one limited fasciectomy and
no more than one dermofasciectomy.³ PNF was only repeated if they relapsed after
a PNF procedure that initially reduced flexion deformity to ≤5°; patients who failed
any PNF procedure (post-operative flexion deformity >5°) and opted for further
surgery received limited fasciectomy rather than PNF.
During the post-trial period modelled, no costs were applied for management of DD
other than interventions (e.g. adalimumab, surgery and outpatient/physiotherapy
consultations associated with surgery), since the costs were negligible within the
trial.
The mean costs and QALYs accrued during the trial period were added to those
estimated in the model to give lifetime costs and QALYs.
Patients with DD were assumed to have higher mortality than the general
population based on a recent data linkage study, but DD stage and treatment were
assumed to have no effect on mortality.¹⁶

TABLE 1
Results of the within-trial economic evaluation from baseline to 12 months

| | | | Mean difference |
|---|---|---|---|
| | | | (95% CI): |
| | Adalimumab | Standard care | adalimumab |
| | Mean (SE) | Mean (SE) | vs standard care |
| N | 70 | 70 | — |
| QALYs^{$} | 0.875 (0.012) | 0.855 (0.012) | 0.004 (−0.019, 0.027) |
| Total NHS & PSS costs baseline | £2070 (53) | £37 (27) | £2035 (1919, 2152) |
| to 12 months (including | | | |
| intervention)* | | | |
| Adalimumab injection costs* | £2030 (43) | £0 (0) | £2028 (1944, 2112) |
| Incremental cost-effectiveness | — | — | £503,410⁺ |
| ratio (ICER): cost per QALY | | | |
| gained | | | |
| Probability of cost-effectiveness | — | — | 0% |
| at willingness to pay threshold | | | |
| of £20,000 per QALY (NHS & | | | |
| PSS perspective)⁺ | | | |

^{$}Differences and p-values derived from linear regression model adjusted for age, site and baseline utility score.
*Differences and p-values derived from linear regression model adjusted for age and site.
The means for each group are unadjusted; the difference between the unadjusted group means will therefore not equal the adjusted treatment effect.
⁺Adalimumab injections provide a small QALY benefit, but are more costly than standard care.
Abbreviations: CI, confidence interval; NHS, National Health Service; PSS, personal and social services; QALY, quality adjusted life year; SE, standard error
Note:
the ICER was generated from un-rounded figures; therefore, the figure cannot be replicated exactly from the rounded figures shown in the table.

TABLE 2
Model inputs estimated on RIDD data. Means and confidence intervals (CI)
represent the average, 2.5^{th} and 97.5^{th} percentiles across 1000
bootstraps (estimated in Microsoft Excel 2016) and include multiple imputation of
flexion deformity, nodule hardness and nodule size. P-values represent the two-
sided bootstrap p-value (2*(1-proportion of bootstraps <0)). The methods of
this analysis are described in eMethods 2, Methods for the analysis of
within-trial data used as inputs for the model section.

| | | Lower | Upper | p- |
|---|---|---|---|---|
| | Mean | 95% CI | 95% CI | value |
| Linear regression predicting QALYs between 6 and 18 months (all participants) | | | | |
| Quiescence at 18 months | 0.0395 | 0.0079 | 0.0704 | 0.018 |
| Baseline EQ-5D utility | 0.5410 | 0.4015 | 0.6887 | |
| Constant | 0.3847 | 0.2581 | 0.5161 | |

Linear regression predicting change in flexion deformity between baseline and 18 months: placebo group

| | | | | |
|---|---|---|---|---|
| Ectopic disease (plantar, Peyronie's | 8.5189 | 2.1683 | 15.3596 | 0.006 |
| or Garrod's knuckle pads) | | | | |
| Constant (Mean change in flexion | 0.8631 | −2.0005 | 4.1074 | |
| deformity for patients without | | | | |
| ectopic disease | | | | |
| Root-mean squared error | 12.6079 | | | |
| Mean change in flexion deformity | 9.3821 | 3.7979 | 15.3832 | |
| for patients with ectopic disease | | | | |
| (coefficient for ectopic disease, | | | | |
| plus constant) | | | | |

Crude proportion of patients in each bootstrap with quiescence or late-stage DD (all participants)

| | | | | |
|---|---|---|---|---|
| Proportion of placebo patients | 0.2149 | 0.1286 | 0.3143 | |
| with late-stage DD at 18 months* | | | | |
| Proportion of placebo patients | 0.2209 | 0.1286 | 0.3429 | |
| quiescent at 18 months* | | | | |
| Proportion of adalimumab patients | 0.1800 | 0.1000 | 0.2857 | |
| with late-stage DD at 18 months* | | | | |
| Proportion of adalimumab patients | 0.3696 | 0.2571 | 0.4857 | |
| quiescent at 18 months* | | | | |
| Relative risk of quiescence with | 1.7902 | 0.9565 | 3.1111 | 0.076 |
| adalimumab vs. placebo | | | | |
| Relative risk of late-stage DD | 0.8881 | 0.4000 | 1.6667 | 0.534 |
| with adalimumab vs. placebo | | | | |

*70 patients were randomized to each treatment group. Since multiple imputation was used to impute missing data on flexion deformity, nodule hardness and nodule size, the number of patients meeting the criteria for quiescence or late stage DD varied between imputed datasets and absolute numbers cannot be given for these proportions.

TABLE 3
Base case results of the model-based economic evaluation. Values
represent means with 95% confidence intervals in brackets.

| | | Standard | 1 course | Repeated courses |
|---|---|---|---|---|
| | | care | adalimumab | adalimumab |
| QALYs^{†} | Trial | 1.279 (1.247, 1.313) | 1.286 (1.248, 1.320) | 1.286 (1.248, 1.320) |
| | Model^{†} | 10.15 (9.80, 10.49) | 10.19 (9.86, 10.51) | 10.45 (10.10, 10.79) |
| | Lifetime^{†} | 11.43 (11.08, 11.78) | 11.48 (11.14, 11.80) | 11.74 (11.40, 12.08) |
| NHS costs^{†} | Trial | £307 (£134, £514) | £2,136 (£1,998, £2,277) | £2,136 (£1,998, £2,277) |
| | Model^{†} | £1,416 (£1,056, £1,887) | £1,333 (£993, £1,780) | £4,071 (£2,600, £6,379) |
| | Lifetime^{†} | £3,552 (£3,165, £4,036) | £5,298 (£4,782, £5,820) | £8,036 (£6,455, £10,469) |

| | | | |
|---|---|---|---|
| Life expectancy | 22.6 (22.0, 23.2) | 22.6 (22.0, 23.2) | 22.6 (22.0, 23.2) |
| Years with early- | 9.71 (6.38, 12.05) | 10.39 (7.23, 12.72) | 13.25 (10.52, 15.61) |
| stage DD | | | |
| Number of courses of | 0.0 (0.0, 0.0) | 1.0 (n1.0, 1.0) | 3.65 (2.35, 5.74) |
| treatment for early- | | | |
| stage DD | | | |
| Number of operations | 1.36 (1.0, 1.88) | 1.30 (0.94, 1.79) | 0.93 (0.63, 1.32) |
| for late-stage DD | | | |
| Incremental QALYs vs. | — | 0.048 (−0.053, 0.166) | 0.307 (0.110, 0.524)* |
| standard care^{†} | | | |
| Incremental cost vs. | — | £1,746 (£1,418, £2,060)* | £4,484 (£2,969, £6,859)* |
| standard care^{†} | | | |
| Cost/QALY vs. | — | £36,125 (dominated, £396,960) | £14,593 (£7,534, £42,698)* |
| standard care^{†} | | | |

*p < 0.05
^{†}Discounted at 3.5% per annum
Abbreviations: DD, Dupuytren's disease; QALY, quality-adjusted life-year.

### EXAMPLE 1 REFERENCES

1. Lanting R, Broekstra D C, Werker P M N, et al. A systematic review and meta-analysis on the prevalence of Dupuytren disease in the general population of Western countries. Plast Reconstr Surg 2014; 133:593-603.
2. Shih B, Bayat A. Scientific understanding and clinical management of Dupuytren disease. Nat Rev Rheumatol 2010; 6:715-26.
3. Chan J K-K, Werker P M N, Nanchahal J. Dupuytren's Disease. Neligan's Plastic Surgery 5th edition, Volume 6 Hand and Upper Limb: Elsevier 2022.
4. Brazzelli M, Cruickshank M, Tassie E, et al. Collagenase Clostridium histolyticum for the treatment of dupuytren's contracture: Systematic review and economic evaluation. Health Technology Assessment 2015; 19:1-201.
5. Ball C, Izadi D, Verjee L S, et al. Systematic review of non-surgical treatments for early dupuytren's disease. BMC Musculoskelet Disord 2016; 17:345.
6. Verjee L S, Verhoekx J S, Chan J K, et al. Unraveling the signaling pathways promoting fibrosis in Dupuytren's disease reveals TNF as a therapeutic target. Proc Natl Acad Sci USA 2013; 110: E928-37.
7. Izadi D, Layton T B, Williams L, et al. Identification of TNFR2 and IL-33 as therapeutic targets in localized fibrosis. Sci Adv 2019; 5: eaay0370.
8. Nanchahal J, Ball C, Davidson D, et al. Anti-Tumour Necrosis Factor Therapy for Dupuytren's Disease: A Randomised Dose Response Proof of Concept Phase 2a Clinical Trial. EBioMedicine 2018; 33:282-88.
9. Nanchahal J, Ball C, Rombach I, et al. Anti-tumour necrosis factor therapy for early-stage Dupuytren's disease (RIDD): a phase 2b, randomised, double-blind, placebo-controlled trial. Lancet Rheumatology 2022; 4: e407-16.
10. Dritsaki M, Rivero-Arias O, Gray A, et al. What do we know about managing Dupuytren's disease cost-effectively? BMC Musculoskelet Disord 2018; 19:34.
11. Grutters J P C, Govers T, Nijboer J, et al. Problems and Promises of Health Technologies: The Role of Early Health Economic Modeling. Int J Health Policy Manag 2019; 8:575-82.
12. Png M E, Dritsaki M, Gray A, et al. Economic evaluation plan of a randomised controlled trial of intra-nodular injection of anti-TNF and placebo among patients with early Dupuytren's disease: Repurposing Anti-TNF for Treating Dupuytren's Disease (RIDD). Wellcome Open Res 2018; 3:156.
13. Nanchahal J, Ball C, Swettenham J, et al. Study protocol: A multi-centre, double blind, randomised, placebo-controlled, parallel group, phase II trial (RIDD) to determine the efficacy of intra-nodular injection of anti-TNF to control disease progression in early Dupuytren's disease, with an embedded dose response study. Wellcome Open Res 2017; 2:37.
14. National Institute for Health and Care Excellence. NICE health technology evaluations: the manual. www.nice.org.uk/process/pmg36/resources/nice-health-technology-evaluations-the-manual-pdf-72286779244741 (date last accessed 7 Feb. 2022).
15. Yoon A P, Kane R L, Hutton D W, et al. Cost-effectiveness of Recurrent Dupuytren Contracture Treatment. JAMA Netw Open 2020; 3: e2019861.
16. Kuo R Y L, Ng M, Prieto-Alhambra D, et al. Dupuytren's Disease Predicts Increased All-Cause and Cancer-Specific Mortality: Analysis of a Large Cohort from the U.K. Clinical Practice Research Datalink. Plast Reconstr Surg 2020; 145: 574e-82e.
17. Alser O, Craig R S, Lane J C E, et al. Serious complications and risk of re-operation after Dupuytren's disease surgery: a population-based cohort study of 121,488 patients in England. Sci Rep 2020; 10:16520.
18. Strong M, Oakley J E, Brennan A. Estimating multiparameter partial expected value of perfect information from a probabilistic sensitivity analysis sample: a nonparametric regression approach. Med Decis Making 2014; 34:311-26.
19. National Institute of Health and Care Excellence (NICE). Radiation therapy for early Dupuytren's disease: Interventional procedure consultation document [IPG573]. www.nice.org.uk/guidance/ipg573 (date last accessed 9 Feb. 2021).
20. Costas B, Coleman S, Kaufman G, et al. Efficacy and safety of collagenase Clostridium histolyticum for Dupuytren disease nodules: a randomized controlled trial. BMC Musculoskelet Disord 2017; 18:374.
21. Walters S J, Brazier J E. Comparison of the minimally important difference for two health state utility measures: EQ-5D and SF-6D. Qual Life Res 2005; 14:1523-32.
22. Drugs.com. Humira Prices, Coupons and Patient Assistance Programs. www.drugs.com/price-guide/humira (date last accessed 21 Jul. 2022).
23. Lanting R, van den Heuvel E R, Werker P M. Clusters in Short-term Disease Course in Participants With Primary Dupuytren Disease. J Hand Surg Am 2016; 41:354-61.
24. Ketchum L D, Donahue T K. The injection of nodules of Dupuytren's disease with triamcinolone acetonide. J Hand Surg Am 2000; 25:1157-62.
25. Yin C Y, Yu H M, Wang J P, et al. Long-term follow-up of Dupuytren disease after injection of triamcinolone acetonide in Chinese patients in Taiwan. J Hand Surg Eur Vol 2017; 42:678-82.
26. Kadhum M, Smock E, Khan A, et al. Radiotherapy in Dupuytren's disease: a systematic review of the evidence. J Hand Surg Eur Vol 2017; 42:689-92.
27. Office for National Statistics. National life tables: UK. www.ons.gov.uk/peoplepopulationandcommunity/birthsdeathsandmarriages/lifeexpectanci es/datasets/nationallifetablesunitedkingdomreferencetables (date last accessed 17 Jan. 2021).

### Example 2: Supplementary Material for Cost-Effectiveness of Adalimumab for Early-Stage Dupuytren's Disease: An Economic Evaluation Based on a Randomized Controlled Trial and Individual-Patient Simulation Model

Additional Methods of within-Trial Analysis

ETABLE 1
Unit cost table for the within-trial economic evaluation

| | Unit cost | Data source |
|---|---|---|
| Trial interventions | | |
| Cost of injection | £352.14 per | The drug used in this trial was Humira 40 mg/0.4 ml solution for |
| | injection | injection pre-filled syringes (AbbVie Ltd) - NHS indicative price |
| | | £704.28, Drug Tariff price £704.28 for two pre-filled syringes. |
| | | Hence, the cost for one trial injection is £352.14. |
| | | Information obtained from https://bnf.nice.org.uk/medicinal- |
| | | forms/adalimumab.html on 28 Jan. 2021. The price NHS hospitals |
| | | pay may vary from this list price, but discounts are confidential. |
| | | Partially used syringes are discarded, i.e. the full drug cost is used |
| | | for each participant. |
| | | (note: at the time of the analysis, Celltrion produced Yuflyma |
| | | 40 mg/0.4 ml solution for injection pre-filled syringes at the same |
| | | drug tariff as described for AbbVie above.) |
| | | More diluted preparations cannot be used for this trial, as |
| | | demonstrated in the previous phase 2a trial.¹ Recently, Celltrion |
| | | Healthcare UK Ltd also started marketing the same dilution, at the |
| | | same drug tariff cost. |
| | | Other providers only manufacture the drug in different |
| | | formulations, and therefore are unsuitable for the sensitivity |
| | | analysis. |
| | | No costs were applied for the saline injections. |
| | | In sensitivity analyses, we investigated the effect of a range of |
| | | lower adalimumab costs. |
| Administration | £122 | As per the health-economics analysis plan² we assumed that the |
| of injection | | injection would be administered by a clinically qualitied clinician |
| | | at consultant level in the outpatient setting: |
| | | Weighted average of all first and follow-up plastic surgery and |
| | | trauma & orthopaedics non-admitted face-to-face attendances, CL |
| | | (Consultant Led) tab |
| | | This cost is included for the adalimumab arm only, as the usual |
| | | care is no treatment. |
| Anaesthetic cream | £1.08 | Ametop 4% gel: an entire tube (1500 mg) is used. NHS indicative |
| | | price: £1.08 for one tube of 1.5 g. This cost was applied where |
| | | indicated in the patient records |
| Hepatitis B test | £4.38 | Required screening before adalimumab treatment. This cost was |
| | | applied to participants in the Adalimumab arm only. Cost based on |
| | | CCG prices. |
| ELiSpot TB screening | £67.61 | Required screening before adalimumab treatment. This cost was |
| | | applied to participants in the Adalimumab arm only. Cost based on |
| | | CCG prices. |
| Hospital attendances | | All costs related to hospital attendances are based on the National |
| | | Schedule of Reference costs - Year 2018-19. |
| | | The ICD-10 code M72.0 - Palmar fascial fibromatosis (Dupuytren) |
| | | was used. |
| Surgeries | | Surgery costs are based on the DC (Day Cases) tab. The costs |
| | | represent the weighted average costs for the procedures listed |
| | | under the relevant HRG codes. |
| Needle fasciotomy | £1,094 | Procedure code: T54.1 - Division of palmar fascia |
| | | HRG code HN45A - Minor Hand Procedures for Non-Trauma, 19 |
| | | years and over |
| Facietcomy | £1,810 | Procedure code: T25.2 - Digital fasciectomy |
| | | HRG: HN44A/HN44B - Intermediate Hand Procedures for Non- |
| | | Trauma, 19 years and over |
| Dermofasciectomy | £2,475 | Procedure code: T56.1 - Dermofasciectomy |
| | | HRG: HN34A/HN34B - Major Hand Procedures for Non-Trauma, |
| | | 19 years and over |
| Splint costs | £37.50 | Participants receive custom-made splints following each surgery |
| | | (excluding radiotherapy). Costs of materials were estimated as |
| | | £11.50 |
| | | Time for preparation of the customized splint was estimated to be |
| | | 20 minutes. An average cost per hour for hospital based scientific |
| | | and professional staff across band 5 to 9 was estimated as £77, |
| | | based on Personal Social Services Research Unit (PSSRU), online |
| | | unit cost database of health and social care professionals |
| | | 2017/2018. |
| | | Total costs associated with the splint are hence estimated as the |
| | | sum of the material cost (£11.50) and staff costs for 20 minutes |
| | | (£26.00) in line with the OTTER trial³. |
| | | This cost is applied to each participant who underwent surgery. |
| Outpatient care | | |
| Hand surgery: | £122 | Weighted average of all first and follow-up plastic surgery and |
| Surgeon consultation | | trauma & orthopaedics non-admitted face-to-face attendances, CL |
| | | (Consultant Led) tab |
| Hand surgery: | £191 | Procedure code: X65.4 - Delivery of a fraction of external beam |
| Radiotherapy | | radiotherapy NEC |
| | | HRG code: SC97Z - Same Day Radiotherapy Admission or |
| | | Attendance (excluding Brachytherapy), OPROC (Outpatient |
| | | Procedure) Tab; weighted average of plastic surgery and trauma & |
| | | orthopaedics |
| Hand surgery: | £136 | Procedure code: S52.1 - Insertion of steroid into subcutaneous |
| Steroid/collagenase | | tissue |
| injection | | HRG code: JC43A - Minor skin procedures, 19 years and over, |
| | | OPROC (Outpatient Procedure) Tab; weighted average of plastic |
| | | surgery and trauma & orthopaedics |
| | | Note: only one participant fell into this category. Based on their |
| | | other trial information and a notes review, they were classed as |
| | | having received a steroid injection, rather than collagenase. |
| Hand surgery: | £101 | Weighted average of all follow-up plastic surgery and trauma & |
| Dressing change | | orthopaedics non-admitted face-to-face attendances, NCL (Non |
| | | Consultant Led) tab |
| Radiology: | £57 | Weighted average costs of ultrasound scans with and without |
| Ultrasound scan | | contrast in an outpatient setting (currency codes RD40Z, RD41Z, |
| | | RD42Z, RD43Z) |
| Physio or hand | £58 | Weighted average of all physio- or hand therapy non-admitted |
| therapy | | face-to-face attendances, NCL (Non Consultant Led) tab |
| Pre-operative | £118 | Some participants specified a pre-operative assessment in their |
| assessment | | questionnaires (free text). In the NHS, these may be appointments |
| | | with a consultant or a nurse, telephone or email assessments. |
| | | Therefore, the appointments are costed as the weighted average of |
| | | all consultant and non-consultant-led non-admitted face-to-face and |
| | | non-face-to-face first and follow-up attendances related to plastic |
| | | surgery and trauma & orthopaedics. CL (Consultant Led) and NCL |
| | | (Non Consultant Led) tabs. |
| X-ray | £22 | Cost for imaging in an outpatient setting, plain film, IMAG |
| | | (diagnostic imaging tab). |
| Primary and | | |
| community care | | |
| GP - appointment at | £39 | Cost per patient contact lasting, on average, 9.22 |
| the GP surgery | | minutes: PSSRU 2019-20 (Chapter 10, page 126). |
| GP - phone call with | £15 | Cost per intervention including other costs: PSSRU 2019-20 |
| GP | | (chapter 10, page 129). Average consultation length of 4 minutes. |
| Practice nurse - | £11 | Personal Social Services Research Unit (PSSRU) unit costs of |
| appointment at GP | | health and social care 2020, table 10.2. £42/hr including |
| surgery | | qualifications. Average consultation lasting 15.5 mins - referenced |
| | | in PSSRU 2015 (based on the 2006/07 UK general practice |
| | | survey). |
| Physiotherapist | £63 | NHS Reference Cost schedule 2018-19, tab CHS, service code |
| | | A08A1 |
| Medication costs | | |
| | Cost per tablet | Costs are based on the British National Formulary published by the |
| | | National Institute for Health and Care Excellence |
| | | (https://bnf.nice.org.uk/, accessed on 28 Jan. 2021) |
| Codeine | £0.04 per tablet | Codeine phosphate 15 mg, NHS indicative price £1.06 for 28 |
| | | tablets. |
| Diclofenac | £0.14 per tablet | Diclofenac potassium 25 mg tablets, NHS indicative price 3.86 for |
| | | 28 tablets. |
| Flucloxacillin | £0.06 per tablet | Flucloxacillin 250 mg capsules, NHS indicative price £1.80 for 28 |
| | | tables. |
| Ibuprofen | £0.02 per tablet | Ibuprofen 200 mg caplets, NHS indicative price £0.25 for 16 |
| | | tablets. |
| Naproxen 500 mg | £0.10 per tablet | Naproxen 500 mg tablets, NHS indicative price £2.86 for 28 tablets. |
| Paracetamol | £0.02 per tablets | Paracetamol 500 mg tablets, NHS indicative price: £0.68 for 32 |
| | | tablets. |
| Paracetamol - codeine | £0.02 per tablets | Co-codamol 8 mg/500 mg caplets, NHS indicative price: £0.70 for |
| | | 32 tablets. |

eMethods 1: Detailed Methods on the within-Trial Economic Evaluation

Full details of the planned analyses have been described in the economic evaluation plan².

Note that the RIDD trial collected data from two sites in the UK (Oxford and Edinburgh), as well as from one site in the Netherlands (Groeningen). In line with the statistical analysis of this trial, only the UK participants are included in this within-trial analysis.

### Data Collection

### Quality of Life

Generic HRQoL was measured using the Euroqol-5 Dimensions-5 Levels questionnaire (EQ-5D-5L). Participants were asked to describe their health over the past 4 weeks in terms of mobility, self-care, usual activities, pain/discomfort and anxiety/depression, and rate any problems with regards to these health states (none, slight, moderate, severe and unable/extreme). Participants also completed a 100-point visual analogue scale (VAS; 0=worst health you can imagine to 100=best health you can imagine). The EQ-5D is a standardized measure of health providing a simple generic measure of health for clinical and economic appraisal.

The EQ-5D-5L was completed at baseline, three, six, nine, 12 and 18 months post randomisation.

Responses to EQ-5D-5L questionnaires were converted into utility scores using the cross-walk to the 3-level version,⁴ using the UK time trade-off tariff. QALYs were calculated using the area under the curve approach, which involves estimating the average EQ-5D utility between each follow-up time, and weighting by survival time. Partially completed EQ-5D-5L questionnaires are considered missing.

### Resource Use

Resource use was collected from patients and trial sites.

The main analyses, a health system perspective i.e. National Health Service (NHS) and personal social services (PSS)) was adopted. The following costs were included in the related costing:

Participants were asked to report on healthcare use for DD in their injected finger. Specifically, participants were asked to report their outpatient hospital use, community healthcare use (GP/nurse appointments, occupational therapists, physiotherapists, and calls to NHS 111 [NHS Direct], medication use, and personal social services (meals on wheels, laundry services, social worker contact, and care worker contact). Data were collected at three, six, nine, 12 and 18 months post randomisation, and covered the period since the last protocol stipulated trial visit. In addition, sites recorded incidence of surgeries in the hand treated in the trial. The type of surgery (Needle fasciotomy, Fasciectomy, Dermofasciectomy, Collagenase) was collected. In line with the patient reported health resource use, surgeries were reported if they occurred in the injected digit.

Additional data were collected on the effect of participants' DD from a societal perspective. Data were collected on the effect on paid work, and financial costs incurred to the participants, their partners, relatives or friends due to their DD in their injected finger.

Participants were also asked about any hospital admissions due to their DD in their injected finger during the trial follow-up. All of these could be matched up to surgeries reported by the trial sites, and were hence not reported separately.

### Methods for Assigning UK-Based Cost Estimates

Cost data were sourced from NHS Reference Costs, the British National Formulary (BNF), Unit Costs of Health and Social Care⁵ or as self-reported by the participants. Details of unit costs used are presented in eTable 1. All unit costs were inflated, where necessary, to 2018-19 prices using the healthcare and community health services inflation index and NHS cost inflation index (NHSCII).⁶

The adalimumab injections were costed at £352.12 per injection (Humira 40 mg/0.4 ml solution, BNF information accessed in January 2021, confirmed accurate in February 2021), plus staff cost at consultant level to facilitate the injection (£122), plus £1.08 for anaesthetic cream (where used). Screening for Hepatitis B and Tuberculosis were also applied for each participants at a cost of £4.38 and £67.61, respectively.

No costs were applied to the saline injections. Costs for injections were applied based on the randomisation allocation, i.e. participants allocated to adalimumab who received saline by accident (one participant) were costed as if they had received adalimumab, and vice versa.

### Missing Data

We followed best practice methods for addressing missing data in cost-effectiveness studies⁷. Missing baseline data were imputed using unconditional mean imputation. Data on receipt of allocated interventions were considered to be complete, i.e. no imputation were performed. For components of resource use where participants provided responses to any questions in the resource diary, we imputed missing values as zero. For example, if a participant indicated that they attended some outpatient services, but left the section on primary and ambulatory care unanswered (i.e. missing), we assumed that no primary and ambulatory services were used. Healthcare resource use was classed as missing if the entire resource use questionnaire was missing, or if the participant did not complete the relevant follow-up.

We used multiple imputation by chained equations to impute missing data on EQ-5D-5L utility scores, and cost components (except costs related to the allocated intervention), at each follow-up time point⁸. Each missing value was imputed as a function of age, recruitment site, and baseline and follow-up EQ-5D-5L score, total NHS costs, injection costs, binary indicator of surgeries performed, change from baseline in nodule area, nodule ferret and flexion deformity in the relevant follow-up tie periods, and whether participants were reported as awaiting surgery at the end of the study. The imputation model was run separately by randomized treatment. We used predictive mean matching to create a total of 50 imputed datasets. We imputed costs and EQ-5D-5L utility score in each period. No deaths were observed, and no corresponding adjustments had to be made to the imputed data. Due to low numbers of surgeries observed especially in the earlier follow-up periods, we imputed data on whether any surgery occurred (rather than if a specific surgery had occurred) for participants who had withdrawn before or during the relevant follow-up time period and therefore data on surgery was missing. The mean cost of all observed surgeries were applied to participants for whom surgeries were imputed. Note: 50 imputations were performed in line with the statistical analysis of the trial, and are not reflective of the amount of missing data observed in this study.

### Within-Trial Analysis

Following multiple imputation, we estimated total costs and QALYs for all participants from the date of study recruitment to 12 months (follow-up period of primary interest), from 12 to 18 months and for the full follow-up period.

We reported descriptive statistics (means, SD as a minimum) for resource use, costs, and EQ-5D-5L utilities at each follow-up time point using only complete data. Differences between arms for the EQ-5D-5L utilities were estimated using multi-level mixed effects linear regression models, to allow for multiple follow-ups clustered within participant. The model was adjusted for treatment allocation, an interaction between follow-up time and treatment allocation, age at randomisation and recruitment site, and baseline utility score. QALYs were analysed using analysed using linear regression models adjusted for baseline utilities, age at randomisation and recruitment site. Combined costs were analysed using linear regression models adjusted for age at randomisation and recruitment site, and other outcomes were analysed by unadjusted regression models. Analyses were performed on the imputed datasets, using Rubin's rule to estimate the adjusted mean difference and standard error for each outcome.

Our analysis followed intent-to-treat principles wherein healthcare resource use, costs and EQ-5D scores were analysed according to treatment allocation, regardless of the treatment actually received. We did not discount total costs and QALYs as the time horizon of the analysis of primary interest was 12 months.

We estimated the incremental cost-effectiveness ratio (ICER) by dividing the mean cost difference between adalimumab and standard care by the mean QALY difference.

We estimated the joint uncertainty around incremental total costs and QALYs (i.e. the difference between adalimumab and standard care), and in the cost-effectiveness, by bootstrapping at 1,000 times from each of the 50 imputed datasets (creating at least 50,000 bootstraps), running the estimation model on each bootstrapped dataset and extracting the estimated treatment effects. From these bootstrapped results, we calculated the probability that adalimumab injections were cost-effective compared with standard care at different threshold values per QALY gained and plotted the results on a cost-effectiveness acceptability curve⁹. These were calculated by estimating the proportion of bootstrap replicates with a net monetary benefit (NMB) above 0 for each threshold value, where the NMB was given by the product of the mean difference in QALYs and the threshold value minus the mean difference in costs.

### Sensitivity Analyses

The following sensitivity analyses were performed:
analysis on the per-protocol (PP) population only, including participants who received at least three injections (unless injections were not delivered due to nodule regression), received no surgery during the follow-up, and only received their randomized treatment (i.e. excluding cross-overs)
investigating the effect of lower adalimumab costs, and presenting the ICER for each of these
including only the subset of surgeries that were deemed to be due to progression of the study nodules, and not due to disease in other nodules
including costs for surgeries for participants who were reported to be awaiting surgery at the 18-month study visits (i.e. investigating the impact of potentially delayed surgeries due to Covid-19 or long waiting times)

As a sensitivity analyses, we performed a complete case analysis, including only individuals who provided complete data over the 12 months trial duration.

Summaries for societal costs reported during the trial, including days of work missed, travel costs incurred, help with household tasks, childcare for participants, their partners or friends/relatives, are summarized in the eTable 10. We did not do a sensitivity analysis from a wider perspective since non-NHS costs reported by trial participants were negligible. Subgroup analyses and analyses exploring heterogeneity were not conducted due to the size of the trial.

Additional Tables and Figures from the within-Trial Analysis

ETABLE 2
Data availability over time

| | EQ-5D-5L utility score | | | Health Resource questionnaire | | |
|---|---|---|---|---|---|---|
| | Adalimumab | Saline | Total | Adalimumab | Saline | Total |
| | (N = 70) | (N = 70) | (N= 140) | (N = 70) | (N = 70) | (N= 140) |
| Baseline | 69 (99%) | 69 (99%) | 138 (99%) | n/a | n/a | n/a |
| 3 months | 67 (96%) | 65 (93%) | 132 (94%) | 68 (97%) | 66 (94%) | 134 (96%) |
| 6 months | 64 (91%) | 65 (93%) | 129 (92%) | 65 (93%) | 64 (91%) | 129 (92%) |
| 9 months | 64 (91%) | 65 (93%) | 129 (92%) | 64 (91%) | 65 (93%) | 129 (92%) |
| 12 months | 63 (90%) | 65 (93%) | 128 (91%) | 64 (91%) | 66 (94%) | 130 (93%) |
| 18 months | 65 (93%) | 63 (90h%) | 128 (91%) | 65 (93%) | 64 (91%) | 129 (92%) |

ETABLE 3
EQ-5D-5L utilities and QALYs by treatment
arm (imputed data for 140 UK participants)

| | | | Treatment effect | |
|---|---|---|---|---|
| | Adalimumab | Saline | Mean difference | p- |
| | Mean (SE) | Mean (SE) | (95% CI) | value |
| EQ-5D-5L utility score* | | | | |
| Baseline | 0.877 (0.014) | 0.850 (0.012) | | |
| 3 months | 0.875 (0.013) | 0.854 (0.013) | 0.006 (−0.028, 0.040) | 0.732 |
| 6 months | 0.866 (0.016) | 0.858 (0.015) | −0.007 (−0.043, 0.029) | 0.715 |
| 9 months | 0.884 (0.015) | 0.857 (0.016) | 0.012 (−0.023, 0.048) | 0.493 |
| 12 months | 0.874 (0.017) | 0.848 (0.015) | 0.011 (−0.026, 0.048) | 0.559 |
| 18 months | 0.864 (0.013) | 0.857 (0.015) | −0.008 (−0.044, 0.029) | 0.680 |
| QALYs** | | | | |
| 0 to 12 months | 0.875 (0.012) | 0.855 (0.012) | 0.004 (−0.019, 0.027) | 0.733 |
| 12 to 18 months | 0.433 (0.007) | 0.425 (0.007) | 0.001 (−0.015, 0.017) | 0.876 |
| 0 to 18 months | 1.308 (0.017) | 1.280 (0.017) | 0.005 (−0.031, 0.042) | 0.774 |

*differences and p-values derived from mixed effects model adjusted for baseline utility, age and site, using a treatment and time interaction
**differences and p-values derived from linear regression model adjusted for baseline utility, age and site

ETABLE 4
EQ-5D-5L utilities and QALYs by treatment arm (available data)

| | | | | | | | | | Treatment effect | |
|---|---|---|---|---|---|---|---|---|---|---|
| | Adalimumab | | | | Saline | | | | Mean difference | |
| | N | Mean (SD) | Median (IQR) | Range | N | Mean (SD) | Median (IQR) | Range | (95% CI)* | p-value |
| EQ-5D-5L | | | | | | | | | | |
| utility score | | | | | | | | | | |
| Baseline | 69 | 0.877 (0.118) | 0.837 | 0.397, 1.000 | 69 | 0.849 (0.102) | 0.837 (0.795, 0.906) | 0.604, 1.000 | | |
| | | | (0.806, 1.000) | | | | | | | |
| 3 months | 67 | 0.876 (0.108) | 0.837 | 0.659, 1.000 | 65 | 0.855 (0.104) | 0.837 (0.795, 1.000) | 0.497, 1.000 | 0.002 | 0.891 |
| | | | (0.795, 1.000) | | | | | | (−0.031, 0.036) | |
| 6 months | 64 | 0.873 (0.126) | 0.837 | 0.548, 1.000 | 65 | 0.857 (0.124) | | 0.555, 1.000 | 0.002 | 0.897 |
| | | | (0.782, 1.000) | | | | | | (−0.032, 0.036) | |
| 9 months | 64 | 0.886 (0.117) | 0.863 | 0.548, 1.000 | 65 | 0.859 (0.127) | 0.837 (0.768, 1.000) | 0.550, 1.000 | 0.011 | 0.530 |
| | | | (0.837, 1.000) | | | | | | (−0.023, 0.045) | |
| 12 months | 63 | 0.875 (0.124) | 0.837 | 0.555, 1.000 | 65 | 0.847 (0.118) | 0.837 (0.768, 1.000) | 0.567, 1.000 | 0.011 | 0.525 |
| | | | (0.795, 1.000) | | | | | | (−0.023, 0.045) | |
| 18 months | 65 | 0.868 (0.102) | 0.837 | 0.642, 1.000 | 63 | 0.858 (0.120) | 0.837 (0.767, 1.000) | 0.587, 1.000 | −0.004 | 0.809 |
| | | | (0.795, 1.000) | | | | | | (−0.038, 0.030) | |
| EQ-5D VAS | | | | | | | | | | |
| Baseline | 70 | 87.3 (9.8) | 90 (85, 95) | 55, 100 | 69 | 88.9 (8.1) | 90 (85, 95) | 70, 100 | | |
| 3 months | 67 | 89.0 (8.1) | 90 (85, 95) | 65, 100 | 66 | 88.0 (9.4) | 90 (80, 95) | 60, 100 | 2.3 (−0.4, 4.9) | 0.100 |
| 6 months | 64 | 87.5 (11.3) | 90 (80, 95) | 45, 100 | 65 | 86.8 (11.6) | 90 (80, 95) | 40, 100 | 2.0 (−0.7, 4.7) | 0.145 |
| 9 months | 64 | 89.3 (9.8) | 91 (85, 95) | 60, 100 | 66 | 90.1 (7.5) | 91 (85, 95) | 65, 100 | 0.5 (−2.2, 3.2) | 0.702 |
| 12 months | 64 | 87.3 (10.9) | 90 (80, 95) | 60, 100 | 66 | 89.0 (10.8) | 92 (85, 96) | 50, 100 | −0.3 (−3.0, 2.4) | 0.810 |
| 18 months | 64 | 88.0 (10.9) | 90 (83, 95) | 50, 100 | 63 | 88.8 (9.7) | 90 (84, 95) | 60, 100 | 0.7 (−2.1, 3.4) | 0.635 |
| QALYs* | | | | | | | | | | |
| 0 to 12 | 58 | 0.883 (0.097) | 0.896 | 0.635, 1.000 | 62 | 0.854 (0.097) | 0.862 (0.788, 0.925) | 0.559, 1.000 | 0.007 | 0.536 |
| months | | | (0.824, 0.959) | | | | | | (−0.016, 0.031) | |
| 12 to 18 | 63 | 0.434 (0.052) | 0.417 | 0.318, 0.499 | 62 | 0.425 (0.054) | 0.417 (0.396, 0.458) | 0.297,0.499 | 0.001 | 0.871 |
| months | | | (0.396, 0.499) | | | | | | (−0.015, 0.017) | |
| 0 to 18 | 58 | 1.322 (0.143) | 1.302 | 0.953, 1.499 | 60 | 1.280 (0.147) | 1.282 (1.187, 1.386) | 0.879, 1.499 | 0.012 | 0.526 |
| months | | | (1.239, 1.458) | | | | | | (−0.026, 0.050) | |

*mean-imputed baseline data were used in the statistical model. Observed data are displayed in the summary statistics.
*QALYs were calculated only where EQ-5D-5D utility scores were available for all timepoints within the relevant follow-up period.

ETABLE 5
Resource use by treatment arm (available data)

| | Adalimumab | Saline | Total |
|---|---|---|---|
| | (n = 70) | (n = 70) | (n = 70) |
| Number of injections per participant | | | |
| no injections received | 0 (0%) | 0 (0%) | 0 (0%) |
| at least 1 injection received | 70 (100%) | 70 (100%) | 140 (100%) |
| at least 2 injections received | 69 (99%) | 64 (91%) | 133 (95%) |
| at least 3 injections received | 64 (91%) | 64 (91%) | 128 (91%) |
| at least 4 injections received | 57 (81%) | 60 (86%) | 117 (84%) |
| Participants who received injections | | | |
| at each follow-up time point | | | |
| baseline | 70 (100%) | 70 (100%) | 140 (100%) |
| 3 months | 68 (97%) | 64 (91%) | 132 (94%) |
| 6 months | 63 (90%) | 63 (90%) | 126 (90%) |
| 9 months | 59 (84%) | 61 (87%) | 120 (86%) |
| Number of injections received | | | |
| 1 | 1 (1%) | 6 (9%) | 7 (5%) |
| 2 | 5 (7%) | 0 (0%) | 5 (4%) |
| 3 | 7 (10%) | 4 (6%) | 11 (8%) |
| 4 | 57 (81%) | 60 (86%) | 117 (84%) |
| Surgeries received during follow-up | | | |
| baseline to 3 months | | | |
| none | 69 (99%) | 69 (99%) | 138 (99%) |
| data unavailable* | 1 (1%) | 1 (1%) | 2 (1%) |
| n3 to 6 months | | | |
| none | 68 (97%) | 65 (93%) | 133 (95%) |
| data unavailable* | 2 (3%) | 5 (7%) | 7 (5%) |
| 6 to 9 months | | | |
| none | 65 (93%) | 64 (91%) | 129 (92%) |
| fasciectomy | 1 (1%) | 0 (0%) | 1 (1%) |
| data unavailable* | 4 (6%) | 6 (9%) | 10 (7%) |
| 9 to 12 months | | | |
| none | 65 (93%) | 62 (89%) | 127 (91%) |
| needle fasciotomy | 0 (0%) | 1 (1%) | 1 (1%) |
| data unavailable* | 5 (7%) | 7 (10%) | 12 (9%) |
| 12 to 18 months | | | |
| none | 63 (90%) | 57 (81%) | 120 (86%) |
| needle fasciotomy | 1 (1%) | 0 (0%) | 1 (1%) |
| fasciectomy | 1 (1%) | 5 (7%) | 6 (4%) |
| dermofasciectomy | 0 (0%) | 1 (1%) | 1 (1%) |
| Type of surgery unknown** | 0 (0%) | 1 (1%) | 1 (1%) |
| data unavailable* | 5 (7%) | 6 (9%) | 11 (8%) |
| Surgeries performed | | | |
| throughout the trial | | | |
| needle fasciotomy | 1 (1%) | 1 (1%) | 2 (1%) |
| fasciectomy | 2 (3%) | 5 (7%) | 7 (5%) |
| dermofasciectomy | 0 (0%) | 1 (1%) | 1 (1%) |
| Type of surgery unknown** | 0 (0%) | 1 (1%) | 1 (1%) |

Community healthcare

| | | | |
|---|---|---|---|
| GP - appointment at | | | |
| GP surgery | | | |
| 3 months | 0/68 (0%) | 0/66 (0%) | 0/134 (0%) |
| 6 months | 0/65 (0%) | 0/64 (0%) | 0/129 (0%) |
| 9 months | 1/64 (2%) | 2/65 (3%) | 3/129 (2%) |
| 12 months | 2/64 (3%) | 1/66 (2%) | 3/130 (2%) |
| 18 months | 0/65 (0%) | 1/64 (2%) | 1/129 (1%) |
| GP - phone call | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 1/64 (2%) | 1/129 (1%) |
| GP - home visit | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Practice nurse - appointment | | | |
| at GP surgery | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 1/64 (2%) | 1/129 (1%) |
| Occupational therapist | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Physio- or hand therapy | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 2/65 (3%) | 2/64 (3%) | 4/129 (3%) |
| Call to NHS direct | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Personal social services | | | |
| Meals on wheels | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Laundry services | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Social worker | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Care worker | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Outpatient hospital use | | | |
| Surgeon consultation: NHS | | | |
| 3 months | 0/68 (0%) | 0/66 (0%) | 0/134 (0%) |
| 6 months | 1/65 (2%) | 0/64 (0%) | 1/129 (1%) |
| 9 months | 1/64 (2%) | 1/65 (2%) | 2/129 (2%) |
| 12 months | 1/64 (2%) | 4/66 (6%) | 5/130 (4%) |
| 18 months | 3/65 (5%) | 5/64 (8%) | 8/129 (6%) |
| Surgeon consultation: private | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Radiotherapy: NHS | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 1/64 (2%) | 1/129 (1%) |
| Radiotherapy: private | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Steroid/collagenase injection: | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 1/66 (2%) | 1/130 (1%) |
| 18 months | 0/65 (0%) | 0/64 (0%) | 0/129 (0%) |
| Steroid/collagenase injection: | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Dressing change: NHS | | | |
| 3 months | 0/68 (0%) | 0/66 (0%) | 0/134 (0%) |
| 6 months | 0/65 (0%) | 0/64 (0%) | 0/129 (0%) |
| 9 months | 1/64 (2%) | 0/65 (0%) | 1/129 (1%) |
| 12 months | 0/64 (0%) | 1/66 (2%) | 1/130 (1%) |
| 18 months | 1/65 (2%) | 3/64 (5%) | 4/129 (3%) |
| Dressing change: private | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Ultrasound scan: NHS | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 1/64 (2%) | 1/129 (1%) |
| Ultrasound scan: private | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Physio- or hand therapy: NHS | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 2/65 (3%) | 3/64 (5%) | 5/129 (4%) |
| Physio- or hand therapy: private | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Emergency department: NHS | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Emergency department: private | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |

ETABLE 6
Health service costs over the 18-month trial period (in £) (imputed data)

| | Adalimumab | Saline | Treatment effect | |
|---|---|---|---|---|
| | Mean (SE) | Mean (SE) | Difference (95% CI)* | p-value |
| Total cost of injections | 2030 (43) | 0 (0) | 2028 (1944, 2112) | <0.001 |
| Ambulatory care costs 0-12 months | 14 (10) | 21 (12) | −6 (−37, 25) | 0.694 |
| Surgery costs 0-12 months | 26 (26) | 16 (16) | 14 (−46, 74) | 0.657 |
| Total NHS & PSS cost 0-12 months | 2070 (53) | 37 (27) | 2035 (1919, 2152) | <0.001 |
| Ambulatory care costs 12-18 months | 14 (9) | 73 (33) | −58 (−126, 9) | 0.091 |
| Surgery costs 12-18 months | 43 (31) | 195 (71) | −154 (−307, −2) | 0.047 |
| Total NHS & PSS cost 12-18 months | 57 (39) | 268 (95) | −213 (−417, −9) | 0.041 |
| Total NHS & PSS cost 0-18 months | 2127 (69) | 305 (102) | 1822 (1577, 2068) | <0.001 |

*differences and p-values derived from linear regression model adjusted for baseline utility, age and site. The means (SE) for each group are unadjusted; the difference between the unadjusted group means will therefore not equal the adjusted treatment effect.
Ambulatory costs include primary care visits, outpatient visits and medication costs
Abbreviations: CI—confidence interval; PSS—personal and social services; SE—standard error.

ETABLE 7
Health service costs over the 18-month trial period (in £) (available data)

| | | | | | Saline | | | |
|---|---|---|---|---|---|---|---|---|
| | Adalimumab | | | | | Mean | Median | |
| | n | Mean (SD) | Median (IQR) | Range | n | (SD) | (IQR) | |
| Total cost of injections | 70 | 2030 (362) | 2185 (2185, | 546, 2189 | 70 | 0 (0) | 0 (0, 0) | 0, 0 |
| | | | 2187) | | | | | |
| Ambulatory care cost | 62 | 15 (93) | 0 (0, 0) | 0, 713 | 63 | 24 (103) | 0 (0, 0) | 0, 683 |
| 0-12 months | | | | | | | | |
| Surgery costs 0-12 | 65 | 28 (229) | 0 (0, 0) | 0, 1848 | 68 | 17 (137) | 0 (0, 0) | 0, 1132 |
| months | | | | | | | | |
| Total NHS cost & PSS | 62 | 2187 (294) | 2186 (2185, | 1638, 4201 | 63 | 42 (235) | 0 (0, 0) | 0, 1815 |
| 0-12 months | | | 2188) | | | | | |
| Ambulatory care costs | 65 | 15 (75) | 0 (0, 0) | 0, 461 | 64 | 78 (281) | 0 (0, 0) | 0, 1357 |
| 12-18 months | | | | | | | | |
| Surgery costs 12-18 | 65 | 46 (267) | (0, 0) | 0, 1848 | 67 | 202 (600) | 0 (0, 0) | 0, 2513 |
| months | | | | | | | | |
| Total NHS & PSS cost | 65 | 61 (340) | 0 (0, 0) | 0, 2309 | 64 | 232 (775) | 0 (0, 0) | 0, 3870 |
| 12-18 months | | | | | | | | |
| Total NHS & PSS cost | 62 | 2251 (471) | 2186 (2185, | 1638, 4658 | 61 | 287 (864) | 0 (0, 0) | 0, 4149 |
| 0-18 months | | | 2189) | | | | | |

| | Treatment effect | |
|---|---|---|
| | Difference | |
| | (95% CI)* | p-value |
| Total cost of injections | 2028 (1943, | <0.001 |
| | 2113) | |
| Ambulatory care cost | −7 (−42 28) | 0.688 |
| 0-12 months | | |
| Surgery costs 0-12 | 16 (−48, 80) | 0.625 |
| months | | |
| Total NHS cost & PSS | 2148 (2054, | <0.001 |
| 0-12 months | 2243) | |
| Ambulatory care costs | −62 (−134, 10) | 0.092 |
| 12-18 months | | |
| Surgery costs 12-18 | −152 (−313, 9) | 0.064 |
| months | | |
| Total NHS & PSS cost | −174 (−384, 35) | 0.102 |
| 12-18 months | | |
| Total NHS & PSS cost | 1965 (1714, | <0.001 |
| 0-18 months | 2215) | |

*differences and p-values derived from linear regression model adjusted for baseline utility, age and site. The means (SE) for each group are unadjusted; the difference between the unadjusted group means will therefore not equal the adjusted treatment effect.
Ambulatory costs include primary care visits, outpatient visits and medication costs
Abbreviations: Cl - confidence interval; PSS - personal and social services; SE - standard error.
Abbreviations: QALY - quality adjusted life year

ETABLE 8
Sensitivity analyses

| | | | Difference |
|---|---|---|---|
| | | | (Adalimumab |
| | | | vs Saline) |
| | Adalimumab | Saline | Mean difference |
| | Mean (SE) | Mean (SE) | (95% CI) |
| ITT population (0-18 months) | | | |
| N | 70 | 70 | — |
| QALYs^{$} | 1.308 (0.017) | 1.280 (0.017) | 0.005 (−0.031, 0.042) |
| Total NHS & PSS costs baseline to 18 | 2127 (69) | 305 (102) | 1822 (1577, 2068) |
| months (including intervention)* | | | |
| Adalimumab injection costs* | 2030 (43) | 0 (0) | 2028 (1944, 2112) |
| Incremental cost-effectiveness ratio | — | — | −£342,873⁺ |
| (ICER): cost per QALY gained | | | |
| Probability of cost-effectiveness at | — | — | 0% |
| willingness to pay threshold of £20,000 | | | |
| per QALY (NHS & PSS perspective) | | | |
| Per-protocol population (0-12 months) | | | |
| N | 60 | 58 | — |
| QALYs^{$} | 0.884 (0.012) | 0.863 (0.012) | −0.004 (−0.028, 0.019) |
| Total NHS & PSS costs baseline to 12 | 2132 (21) | 0 (0) | 2131 (2088, 2174) |
| months (including intervention)* | | | |
| Adalimumab injection costs* | 2131 (21) | 0 (0) | 2130 (2088, 2172) |
| Incremental cost-effectiveness ratio | — | — | −£476,045 |
| (ICER): cost per QALY gained | | | |
| Probability of cost-effectiveness at | — | — | 0% |
| willingness to pay threshold of £20,000 | | | |
| per QALY (NHS & PSS perspective) | | | |
| Assuming total injection costs of £50 | | | |
| (per injection, including drug and | | | |
| administration), baseline to 12 months | | | |
| N | 70 | 70 | |
| QALYs^{$} | 0.875 (0.012) | 0.855 (0.012) | 0.004 (−0.019, 0.027) |
| Total NHS & PSS costs baseline to 12 | 226 (36) | 38 (27) | 193 (104, 281) |
| months (including intervention)* | | | |
| Adalimumab injection costs* | 186 (4) | 0 (0) | 186 (178, 193) |
| Incremental cost-effectiveness ratio | — | — | £ 47,721 |
| (ICER): cost per QALY gained | | | |
| Probability of cost-effectiveness at | — | — | 31% |
| willingness to pay threshold of £20,000 | | | |
| per QALY (NHS & PSS perspective) | | | |
| including only surgery costs where | | | |
| surgery prompted by study nodule, | | | |
| baseline to 12 months | | | |
| N | 70 | 70 | |
| QALYs^{$} | 0.875 (0.012) | 0.855 (0.012) | 0.004 (−0.019, 0.027) |
| Total NHS & PSS costs baseline to 12 | 2044 (43) | 38 (27) | 2007 (1907, 2107) |
| months (including intervention)* | | | |
| Adalimumab injection costs* | 2030 (43) | 0 (0) | 2028 (1944, 2112) |
| Incremental cost-effectiveness ratio | — | — | £496,390 |
| (ICER): cost per QALY gained | | | |
| Probability of cost-effectiveness at | — | — | 0% |
| willingness to pay threshold of £20,000 | | | |
| per QALY (NHS & PSS perspective) | | | |
| costing surgery for those awaiting | | | |
| surgery at the end of their follow-up | | | |
| N | 70 | 70 | |
| QALYs^{$} | 0.875 (0.012) | 0.855 (0.012) | 0.004 (−0.019, 0.027) |
| Total NHS & PSS costs baseline to 12 | 2070 (53) | 37 (27) | 2035 (1919, 2152) |
| months (including intervention)* | | | |
| Adalimumab injection costs* | 2030 (43) | 0 (0) | 2028 (1944, 2112) |
| Incremental cost-effectiveness ratio | — | — | £503,410 |
| (ICER): cost per QALY gained | | | |
| Probability of cost-effectiveness at | — | — | 0% |
| willingness to pay threshold of £20,000 | | | |
| per QALY (NHS & PSS perspective) | | | |

^{$}differences and p-values derived from linear regression model adjusted for age, site and baseline utility score.
*differences and p-values derived from linear regression model adjusted for age and site.
The means for each group are unadjusted; the difference between the unadjusted group means will therefore not equal the adjusted treatment effect.
⁺Adalimumab injections provide a small QALY benefit, but are more costly than standard care.
Abbreviations: CI—confidence interval; PSS—personal and social services; QALY—quality adjusted life year; SE—standard error
Note:
the ICER was generated from un-rounded figures, meaning that the figure cannot be replicated exactly from the rounded figures shown in the table.

ETABLE 9
Considering the impact of different injections costs on the total NHS & PSS costs, baseline to 12 months

| | Adalimumab | | Saline | | Cost difference (£) | | | | | |
|---|---|---|---|---|---|---|---|---|---|---|
| Cost per | Total cost (£) | | Total cost (£) | | | Lower 95% | Upper 95% | | QALY | |
| injection (£) | Mean | SE | Mean | SE | Mean | CI limit | CI limit | p-value | mean | ICER |
| 0 | £40 | £37 | £37 | £27 | £7 | −£81 | £96 | 0.869 | 0.004 | £1,833 |
| 10 | £77 | £37 | £37 | £27 | £45 | −£44 | £133 | 0.323 | 0.004 | £11,011 |
| 20 | £115 | £36 | £37 | £27 | £82 | −£7 | £170 | 0.071 | 0.004 | £20,188 |
| 30 | £152 | £36 | £37 | £27 | £119 | £30 | £207 | 0.009 | 0.004 | £29,366 |
| 40 | £189 | £36 | £37 | £27 | £156 | £67 | £244 | 0.001 | 0.004 | £38,543 |
| 50 | £226 | £36 | £37 | £27 | £193 | £104 | £281 | <0.001 | 0.004 | £47,721 |
| 60 | £263 | £36 | £37 | £27 | £230 | £142 | £318 | <0.001 | 0.004 | £56,898 |
| 70 | £300 | £36 | £37 | £27 | £267 | £179 | £356 | <0.001 | 0.004 | £66,076 |
| 80 | £337 | £36 | £37 | £27 | £304 | £216 | £393 | <0.001 | 0.004 | £75,253 |
| 90 | £375 | £36 | £37 | £27 | £341 | £253 | £430 | <0.001 | 0.004 | £84,431 |
| 100 | £412 | £36 | £37 | £27 | £378 | £290 | £467 | <0.001 | 0.004 | £93,609 |
| 110 | £449 | £37 | £37 | £27 | £416 | £327 | £504 | <0.001 | 0.004 | £102,786 |
| 120 | £486 | £37 | £37 | £27 | £453 | £364 | £542 | <0.001 | 0.004 | £111,964 |
| 130 | £523 | £37 | £37 | £27 | £490 | £401 | £579 | <0.001 | 0.004 | £121,141 |
| 140 | £560 | £37 | £37 | £27 | £527 | £437 | £616 | <0.001 | 0.004 | £130,319 |
| 150 | £597 | £37 | £37 | £27 | £564 | £474 | £654 | <0.001 | 0.004 | £139,496 |
| 160 | £635 | £37 | £37 | £27 | £601 | £511 | £691 | <0.001 | 0.004 | £148,674 |
| 170 | £672 | £37 | £37 | £27 | £638 | £548 | £728 | <0.001 | 0.004 | £157,851 |
| 180 | £709 | £38 | £37 | £27 | £675 | £585 | £766 | <0.001 | 0.004 | £167,029 |
| 190 | £746 | £38 | £37 | £27 | £712 | £622 | £803 | <0.001 | 0.004 | £176,206 |
| 200 | £783 | £38 | £37 | £27 | £749 | £658 | £841 | <0.001 | 0.004 | £185,384 |
| 210 | £820 | £38 | £37 | £27 | £787 | £695 | £878 | <0.001 | 0.004 | £194,561 |
| 220 | £857 | £38 | £37 | £27 | £824 | £732 | £916 | <0.001 | 0.004 | £203,739 |
| 230 | £895 | £39 | £37 | £27 | £861 | £768 | £953 | <0.001 | 0.004 | £212,916 |
| 240 | £932 | £39 | £37 | £27 | £898 | £805 | £991 | <0.001 | 0.004 | £222,094 |
| 250 | £969 | £39 | £37 | £27 | £935 | £842 | £1,028 | <0.001 | 0.004 | £231,272 |
| 260 | £1,006 | £40 | £37 | £27 | £972 | £878 | £1,066 | <0.001 | 0.004 | £240,449 |
| 270 | £1,043 | £40 | £37 | £27 | £1,009 | £915 | £1,104 | <0.001 | 0.004 | £249,627 |
| 280 | £1,080 | £40 | £37 | £27 | £1,046 | £951 | £1,141 | <0.001 | 0.004 | £258,804 |
| 290 | £1,117 | £41 | £37 | £27 | £1,083 | £988 | £1,179 | <0.001 | 0.004 | £267,982 |
| 300 | £1,155 | £41 | £37 | £27 | £1,121 | £1,024 | £1,217 | <0.001 | 0.004 | £277,159 |
| 310 | £1,192 | £41 | £37 | £27 | £1,158 | £1,061 | £1,254 | <0.001 | 0.004 | £286,337 |
| 320 | £1,229 | £42 | £37 | £27 | £1,195 | £1,097 | £1,292 | <0.001 | 0.004 | £295,514 |
| 330 | £1,266 | £42 | £37 | £27 | £1,232 | £1,134 | £1,330 | <0.001 | 0.004 | £304,692 |
| 340 | £1,303 | £43 | £37 | £27 | £1,269 | £1,170 | £1,368 | <0.001 | 0.004 | £313,869 |
| 350 | £1,340 | £43 | £37 | £27 | £1,306 | £1,207 | £1,405 | <0.001 | 0.004 | £323,047 |

ETABLE 10
Exploration of non-NHS costs - percentage of participants
who reported any of the relevant costs

| | Adalimumab | Placebo | Total |
|---|---|---|---|
| Missed work - participant | | | |
| 3 months | 0/68 (0%) | 1/66 (2%) | 1/134 (1%) |
| 6 months | 1/65 (2%) | 0/64 (0%) | 1/129 (1%) |
| 9 months | 1/64 (2%) | 0/65 (0%) | 1/129 (1%) |
| 12 months | 0/64 (0%) | 1/66 (2%) | 1/130 (1%) |
| 18 months | 2/65 (3%) | 1/64 (2%) | 3/129 (2%) |
| Missed work - partner | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Missed work - relative/partner | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Travel cost - participant | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 2/64 (3%) | 2/129 (2%) |
| Travel cost - partner | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 1/65 (2%) | 0/64 (0%) | 1/129 (1%) |
| Travel cost - relative/partner | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 1/64 (2%) | 1/129 (1%) |
| Child care - participant | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Child care - partner | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Child care - relative/partner | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Home work - participant | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Home work - partner | | | |
| 3 months | 0/68 (0%) | 0/66 (0%) | 0/134 (0%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |
| Home work - relative/partner | | | |
| 3 months | 1/68 (1%) | 0/66 (0%) | 1/134 (1%) |
| 6 months | 0/65 (0%) | 0/64 (0%) | 0/129 (0%) |
| 9 months | 0/64 (0%) | 0/65 (0%) | 0/129 (0%) |
| 12 months | 0/64 (0%) | 0/66 (0%) | 0/130 (0%) |
| 18 months | 0/65 (0%) | 0/64 (0%) | 0/129 (0%) |

### Additional Methods of Model-Based Extrapolation

eMethods 2: Additional Assumptions and Methods of the Model-Based Extrapolation

### More Detailed Description of the Model

### Definitions

Quiescence was defined as having all three of the following:
   nodule area stayed the same or decreased between baseline and 18 months AND
   hardness measured by the standard durometer stayed the same or decreased between baseline and 18 months, AND
   ≤5° increase in active flexion deformity in the joint closest to the study nodule between baseline and 18 months (hyperextension is given a negative flexion deformity value). Including flexion deformity in the definition of quiescence ensures that patients who do have disease progression during the trial are not incorrectly counted as being quiescent. The rationale for using active flexion deformity in this analysis is that (a) inclusion criteria were based on active flexion deformity and (b) active flexion deformity more accurately reflects what the patient can do with their hand.
Late-stage DD was defined as active flexion deformity >30° in the joint closest to the study nodule. In calculating the proportion of patients with late-stage DD by 18 months within the trial population, patients who had had had needle fasciotomy, fasciectomy or dermofasciectomy or surgery of an unknown type during the trial were also counted as having late-stage DD at 18 months.
Ectopic disease is defined as the presence of one or more out of plantar, Peyronie's disease or Garrod's knuckle pads. Patients with ectopic disease have previously been shown to have greater propensity for disease progression, so we tested whether ectopic disease had an impact on the model parameters estimated on the RIDD data.
Treatment success in late-stage DD was defined as a reduction in flexion deformity to ≤5°, while treatment failure was defined as any other outcome from surgery (following Brazzelli et al¹⁰).
Recurrence in late-stage DD was defined as a return to flexion deformity ≥20°, after surgery has reduced flexion deformity to ≤5°, following Brazzelli et al.¹° There is debate in the literature about which cut-off should be used to define recurrence, although we follow the methods of Brazzelli et al.¹⁰ as specified in the health economics analysis plan.²

The base case analysis compared three strategies for managing early-stage DD (sensitivity analyses comparing against steroids and radiotherapy are described in the last section of eMethods 2):
No disease-modifying treatment for early-stage DD. No use of adalimumab at any stage in the disease pathway.
Four initial adalimumab injections at the start of the trial period, with no subsequent use of adalimumab or any treatments for early-stage DD in the model period
Four initial adalimumab injections at the start of the trial period, followed by a further course of four adalimumab injections in quiescent patients as soon as the patient feels that their condition has reactivated and the nodule is starting to get bigger again. No retreatment with adalimumab in patients who were not quiescent following initial treatment. The duration of this quiescence will be a random parameter in the model. No limit was set on the maximum number of treatment courses that patients could have.

The model focuses on the impact of treating (or retreating) one nodule and does not capture the impact of other nodules. Many RIDD participants had more than one nodule, so patients' baseline utility may have been reduced by DD in other joints. Adalimumab would normally need to be administered separately to each nodule, so the cost of treating multiple nodules would be additive, although there is no evidence on the impact that treating multiple nodules would have on quality of life. In clinical practice, the severity of DD in multiple nodules may be taken into account when deciding whether or not to operate in late-stage DD and if surgery is done, all nodules in the same hand would normally be operated on.

A cohort of individual patients with active early-stage DD (based on the placebo arm of the RIDD trial) are followed over time through a sequence of treatments. Treatment for early-stage DD may lead to quiescence (which may be maintained by retreatment). During periods of quiescence, the patient will not experience changes in flexion deformity. By contrast, during periods when the patients are not quiescent, patients will experience progressive changes in flexion deformity that reflect the average change in flexion deformity during the RIDD trial for placebo-treated patients with and without ectopic disease, and the variability between patients in that sample.

As described in the paper and FIG. 1, once patients' flexion deformity which is 30°, they are assumed to have late-stage DD and move between the health states for late-stage DD used by Brazzelli et al.¹⁰ Patients initially enter the untreated state and have a chance of undergoing surgical treatment each cycle. If they have surgery, they will move into either the treatment success state (i.e. surgery reduced flexion deformity to ≤5°) or the treatment failure the following cycle. From the failure state, they have a chance of moving on to the next line of treatment each cycle. From the success state, patients have a chance of recurrence each cycle, at which point they will have the same type of surgery again until the reach the maximum number of tries at that type of surgery, at which point they will move onto the next line of treatment.

The model begins at the end of the 18-month trial (after the initial course of adalimumab). Costs and QALYs accrued during the 18 month trial period were added to those estimated during the model to give lifetime outcomes; this was done by adding the mean costs during the 18 month trial period (with multiple imputation, averaged over 50,000 bootstraps) to the point estimate from the model.

The model used six-month cycles. The time horizon for the analysis was 55 years from randomisation, since this was the general population life expectancy of the youngest UK patient randomized to placebo.¹¹) The costs and QALYs of each trial participant were estimated until the individual died, or until 55 years from randomisation (whichever happened sooner).

In the base case analysis, we assumed, based on common treatment pathways¹² that
the first line of surgical treatment for late-stage DD is percutaneous needle fasciotomy (PNF) and that patients will have up to 3 PNFs if each is successful
the second line of surgical treatment for late-stage DD is LF, which may only happen once
the third line of surgical treatment for late-stage DD is DF, which may only happen once
following failure or recurrence of DF, patients will receive “best supportive care” and have no further surgery and remain in the recurrence or failure state indefinitely. This was assumed to not include joint fusion or amputation.

Patients will only have the maximum number of operations of each type if the earlier ones were successful (i.e. reduced flexion deformity to)≤5° and they recurred to flexion deformity ≥20°: any failure (i.e post-surgery flexion deformity)>5° means that patients move up to the next treatment (i.e. if the 2^{nd} fails, then move on to LF). A sensitivity analysis explored the impact of assuming that the treatment pathway for late-stage DD ends after 3 PNFs, since this was the most cost-effective treatment sequence in the analysis by Yoon et al.¹³

Other treatments for early-stage DD (e.g. vitamin a, physiotherapy) were not evaluated in the study because there is insufficient evidence on their efficacy¹⁴ and as these interventions are very rarely used in clinical practice. Collagenase was not included in the model pathway because it has previously been shown to be more costly and no more effective than PNF.¹⁰ Amputation and joint fusion were not included in the model pathways because they occur relatively rarely and we are not aware of any data on the outcomes of treatment or utilities after surgery. Omitting these from the pathway is unlikely to change the conclusions about whether adalimumab is cost-effective because these interventions occur in a very small proportion of patients long after adalimumab treatment has finished.

Costs for each intervention used in the model were based on drug prices,¹⁵ NHS tariffs^{6,16} and published studies¹³ (eTable 11). In early-stage DD, the only NHS costs applied during the model period was for adalimumab injections, since non-treatment costs within the trial period were negligible. In late-stage DD, we included the cost of the procedure itself, outpatient visits, physiotherapy and dressing changes, plus managing hand-related adverse events (eTable 11 and Assumptions section below).

Pre-randomisation utilities for each trial participant were assumed to change over the trial period depending on their age, whether they were quiescent and (once they progressed too late-stage DD) which late-stage DD disease state they were in. EQ-5D utilities were assumed to be 0.0197 (95% CI: 0.0040, 0.0352) higher during any cycle in which patients were quiescent, based on a regression conducted on RIDD participants (see Methods for the analysis of within-trial data used as inputs for the model section below). Each disease state for late-stage DD was associated with a utility decrement, which was based on the values used by Brazelli et al: 10 since Brazelli et al. assumed that treatment success had a utility of 1 (which did not reflect utilities in our sample), we estimated disutilities for recurrence and failure that equalled 1 minus the utilities used by Brazelli et al. Each cycle spent in the failure state was associated with a 0.224 utility decrement (compared with the utility patients would have had if they still had early-stage DD). Each cycle spent in the recurrence state (or the untreated late-stage DD that patients enter when they first develop late-stage DD but have not yet undergone surgery) was associated with a 0.035 utility decrement (compared with the utility patients would have had if they still had early-stage DD). In all cases, utility was assumed to decrease with age based on the equations estimated by Ara and Brazier¹⁷ (see Assumptions section).

A half cycle correction was used to adjust QALYs in the year when patients died, but was not applied to costs, since all treatments were assumed to be given at the beginning of the cycle (see Assumptions section below).

Costs and QALYs after the end of the 18 month trial were discounted at 3.5% per annum, following NICE's reference case.¹⁸ Life expectancy was not discounted. Costs and QALYs accrued during the 18-month trial were not discounted in line with the base case within trial economic evaluation.

### Differences Between the Conceptual Model of the HEAP and the Final Model

The model structure was developed based on a systematic review of previous economic evaluations in DD¹⁹ and extensive discussions with clinicians specializing in the management of DD.

The model differs from that outlined in the HEAP² in the following respects:
The model used individual patient simulation in order to more naturally model the transition from early-stage to late-stage DD and differs slightly from a Markov structure in the way that transitions to late-stage DD occur. The model simulated a cohort of RIDD participants and allowed their individual utilities and flexion deformity to change over time. Flexion deformity changed by different amounts each cycle and when the individual's flexion deformity reaches 30°, they are considered to have late-stage DD. This means that the probability of developing late-stage DD increases with the duration of active early-stage DD and is higher for patients who enter the model with high flexion deformity. By contrast, the HEAP stated that a Markov structure would be used, whereby the probability of progressing to late-stage DD would be either constant over time or vary with time since randomisation. Other than the transition from early-stage to late-stage DD, the model structure mirrors a Markov model with individual patient simulation. Using individual patient simulation also facilitates a great deal of flexibility in treatment sequences, retreatment criteria and allowing for heterogeneity.
“Treatment success” was defined as “quiescence”. No distinction is made between treatment failure and recurrence in early-stage DD, other than the fact that patients with “treatment failure” who do not reach quiescence for initial treatment have three more years with progression of flexion deformity than patients who achieve quiescence lasting three years. Within the final model, quiescence is assumed to last three years for all patients, rather than applying a probability of recurrence each cycle.
Since a 2020 study showing that mortality among 42,000 people with DD was 50% higher than among people without DD,²⁰ we allowed for the excess mortality of DD within the model.

### Additional Assumptions for the Model-Based Extrapolation

Assumptions around disease progression and treatment in early-stage DD:
We used RIDD data to calculate the probability of patients achieving quiescence and the probability of developing late-stage DD during the 18 month trial period (see ‘Methods for the analysis of within-trial data used as inputs for the model’ for details).
Quiescent patients were assumed to have 0% chance of progressing to late-stage DD for X years. In the absence of external data on the duration of quiescence, the base case analysis assumed that quiescence lasts for double the duration of the trial (3 years) regardless of whether quiescence was achieved with adalimumab treatment or without treatment. This was varied in sensitivity analysis between the trial duration (1.5 years) and 5 years (~3 time the trial duration). The lower limit (1.5 years) represents a very conservative estimate, since the trial results show that nodule area and hardness were still decreasing 1.5 years after starting treatment.²¹
Patients who did not reach quiescence with initial treatment were assumed to not receive subsequent courses of treatment.
Patients with late-stage DD at end of trial enter the model in the late-stage DD state (with a history of surgery where appropriate). Regardless of whether they have already had surgery, they will therefore spend at least 6 months in the untreated DD state, before potentially having the 1^{st} surgical procedure applicable to that treatment arm. The RIDD sample is too small to reliably estimate impact of surgery and only one patient had 18 month flexion deformity after surgery. The cost of surgery during the trial period will be captured in the within-trial costs for each study arm.
Data for all patients randomized to placebo in RIDD were used to estimate the rate of change in flexion deformity in patients who were not quiescent but had progressive early-stage DD (see ‘Methods for the analysis of within-trial data used as inputs for the model’ for details).
within the model, patients who are quiescent at the end of the trial are assumed to have end of trial flexion deformity equal to their flexion deformity at randomisation, since only patients with a <5° change in flexion deformity are counted as quiescent.
End of trial flexion deformity for patients who have progressive early-stage DD at the end of the trial was estimated based on the regression predicting change in flexion deformity as a function of ectopic disease.
Within the model, no costs were associated with early-stage DD in the absence of treatment because the costs are negligible within the trial. Costs relating to late-stage DD or surgery for late-stage DD are captured separately within the model and costs accrued in each arm during the trial were added to the costs estimated in the model.
We assumed that the effect of subsequent courses of adalimumab on flexion deformity is the same as was observed for patients' first course during the trial.
No costs or disutilities were applied for treatments for early-stage DD. Within the trial, no treatment-related adverse events were observed other than minor site reactions (itching, redness, bruising, haematoma), which arose more commonly in the saline group.²¹ Steroid injections are associated with complications (including diabetes) and radiotherapy carries a risk of malignancy although there are no data on the magnitude of this risk.^{22,23}

Assumptions regarding patient pathway in late-stage DD:
Patients are considered to move from early-stage to late-stage to DD when their flexion deformity 1st reaches ≥30°.
The switch from early to late-stage DD is assumed to be irreversible for that digit (even if flexion deformity is reduced through surgery, patients cannot go back to the early-stage DD part of the model and/or have early-stage DD treatments)
When patients first move from early-stage DD to late-stage DD, they move into an initial state of “untreated late-stage DD” and have a certain probability of having treatment each year. In the base case analysis, we based the probability of treatment for untreated late-stage DD on Brazzelli's estimate of the proportion of patients having retreatment with PNF.¹⁰
After patients' flexion deformity reaches 30°, all patients will have at least 6 months' delay before undergoing the 1^{st} surgical treatment for late-stage DD
Patients will be retreated with the same intervention for late-stage DD if they have recurrence after initial success (up to the maximum number of repeats for that treatment), but will move to the next treatment if they have any failure. For example, in the base case analysis, where we modelled a treatment pathway where patients had up to 3 PNF operations, followed by up to 1 LF operation, followed by DF, the following treatment pathways would be possible (where patients move between states every 6 months):
   PNF, success, recurrence, PNF, success, recurrence, PNF, success, recurrence, LF, recurrence, DF;
   PNF, success, recurrence, PNF, failure, LF, success, recurrence, DF
   However, these pathways were not permitted because once patients fail one type of surgery they will not receive it again and as patients cannot move back to earlier treatments:
      PNF, failure, PNF
      PNF, failure, LF, failure, PNF
We assumed that no patients with late-stage DD would have steroids, radiotherapy or adalimumab. However, we did include the cost of physiotherapy, outpatient visits and dressing changes around the time of surgery.
Late-stage DD patients will always have at least a year's gap between any 2 operations if they fail treatment (the same as the Brazzelli¹⁰ model).
Late-stage DD patients will always have at least 6 months' gap between recurrence and subsequent retreatment (this is the same as the Brazzelli¹⁰ model)
When patients are in the recurrence, failure or untreated late-stage DD states, there is a probability of them undergoing surgery each cycle. The probability of undergoing treatment is assumed to vary depending on what treatment they are going to happen next, but is assumed to be independent of what treatment may have had previously. [The model has separate parameters for the probability of having the 1^{st} surgery of type X from the failure/untreated state and for the probability of having retreatment with X from the recurrence state, but in the base case analysis these are set to the same]
Treatment is assumed to be the first thing that happens in each cycle. Retreatment criteria are applied based on the flexion deformity at the end of the last cycle.
We assumed that the probability of success (i.e. correction of flexion deformity) with DF is the same as for LF, since Bainbridge et al. found that the mean number of Tubiana stages that patients improved was very similar for LF and DF.²⁴ We assumed that treatment for early-stage DD has no impact on the outcomes of late-stage DD treatment (except to potentially delay progression to late-stage DD).
A proportion of patients undergoing surgery for late-stage DD were assumed to have physiotherapy, outpatient visits and/or dressing changes. Since the Brazzelli et al used expert opinion to elicit the number of these consultations,¹⁰ we used the mean number of such consultations among the RIDD participants undergoing surgery; although these are based on very small numbers of patients, they represent the only available UK data that we are aware of.
Each operation for late-stage DD has a probability of intraoperative adverse events.²⁵ The analysis considered only hand-related complications from late-stage DD surgery that lead to hospital intervention and specifically excluded the impact on cardiovascular events associated with late-stage DD surgery,²⁵ loss of hand function.²⁶ Following Brazzelli et al, intraoperative adverse events lead to additional costs and a temporary decrease in quality of life.¹⁰
We assumed that the procedure cost and the probability of failure/recurrence/complications is unaffected by previous surgery (as assumed previously). This assumption is supported by the Mendelar study,²⁷ which found no difference in extension deficit or Michigan hand score between 1^{st} and subsequent operations.

Assumptions around mortality in late-stage and early-stage DD:
Mortality rates during the modelled period were based on all-cause mortality rates for the general population,¹¹ multiplied by published hazard ratios showing mortality for patients with DD, compared with those with no DD.²⁰
Data on all-cause mortality for the general population in 2017-2019 was obtained from the Office of National Statistics.¹¹ The mortality rates (mₓ) from the United Kingdom life table 2017-19 for each individual year of age were applied to each trial participant based on their age and gender: for example, a man randomized at age 30 were subjected to the mortality rate for 35-year-old men during the fifth year after randomisation, whereas a woman randomized at age 50 was subjected to the mortality rate for 55-year-old woman during the fifth year after randomisation.
Mortality rates for patients aged between 101 and 119 were assumed to equal mortality at age 100.
All patients who reached 120 years of age were assumed to die that year to ensure that the model did not project implausible life expectancies.
The hazard ratio for DD vs no DD from a UK database linkage study²⁰ was used to adjust annual mortality data¹¹ for each patient using the life table methods of Pharaoh and Hollingsworth.²⁸ The excess mortality for DD was assumed to apply to both early-stage DD and late-stage DD. For simplicity, we assumed that the hazard ratio for mortality with DD versus no DD was constant over time and with age and that the prevalence of DD was constant; although there is evidence that people with DD actually have lower mortality during the 1^{st} 12 years after diagnosis and higher mortality after that this,²⁰ the impact on treatment on mortality is not known and could not be captured in the model. We used the unadjusted hazard ratio from Kuo (1.48; 99% CI, 1.29 to 1.70) because the available life tables are not adjusted for risk factors. These simplifications are unlikely to have any significant effect on the results because the hazard ratio is simply used to estimate the length of time that each patient spends in the model. In addition to there being no evidence on how or whether mortality differs between early-stage and late-stage DD, it was important to assume no difference in mortality to avoid assuming that treatments that reduce flexion deformity in early-stage DD would have a mortality benefit, which cannot be concluded from the evidence at present. By contrast, assuming a constant hazard ratio for all patients means that the excess mortality for DD simply reduces the amount of time that patients on all treatments accrue costs and quality-of-life.
We added the QALYs accrued in the trial to those projected by the model; since no patients died during the trial period, the lifetime projections incorporate no mortality during the first 18 months after randomisation.
In principle, there could be interactions between DD treatments and interventions for other conditions that affect life expectancy or that have non-additive effects on quality of life, although these are beyond the scope of this analysis since such interactions are likely to affect only a minority of patients. For simplicity, we assumed that life expectancy, quality of life impact of other conditions and the excess mortality for DD will remain constant in the future and that interventions that may affect life expectancy or the quality of life impact from other conditions are adopted based on standard decision rules.
UK life tables were based on pre-COVID data (2017-19)¹¹ and no adjustment was made to estimate the impact that Covid may have on mortality rates in the future, since the long-term impact of the virus is not yet known.
Surgery for late-stage DD and injections for early-stage DD were assumed to always occur at the beginning of the six month cycle, other than adalimumab, which was assumed to be given at the beginning of the cycle and exactly half way through the cycle. No half cycle correction was therefore applied for treatment costs. All other costs captured in the model (e.g. outpatient visits, physiotherapy, dressing changes and time off work) are likely to arise around the time of surgery, so were also applied in full if the patient died within six months of surgery. For quarterly injections (such as adalimumab), we applied the cost of 1.5 injections in the cycle in which the patient died, based on the assumption that all patients dying within cycle c will have the first injection and half of them will have the second.
A half-cycle correction was applied for QALYs: in the cycle when patients died, they were assumed to accrue half of the QALYs and half of the non-NHS costs that they would otherwise have accrued.

Assumptions around utilities and QALYs:
While they have early-stage DD, patients' quality-of-life will be based on that individual's pre-randomisation EQ-5D utility, minus any age-related decline in quality-of-life and plus improvement in QoL due to quiescence. Utilities in late-stage DD were based on the values used by Brazzelli et al.¹⁰ The QALYs accrued during the trial were added to model outcomes to give lifetime QALYs.
An improvement in quality of life compared with baseline was applied to all cycles spent in the quiescent state; this was based on a regression predicting QALYs accrued between month 6 and month 18 in RIDD, with multiple imputation of missing post-baseline data (see Methods for the analysis of within-trial data used as inputs for the model section). Quality-of-life at the end of each cycle was based on whether the patient was quiescent on the last day of that cycle. In the cycle during which quiescence ends, patients' quality-of-life was assumed to linearly revert to their pre-randomisation quality-of-life (minus any age modifier). For example, if the duration of quiescence is between 3.0 and 3.49 years, the full QoL benefit of quiescence will be applied during cycles 1-3; during cycle 4, patients will get half of the QoL benefit of quiescence. If patients are retreated as soon as quiescence ends, they are assumed to continue to experience the quality-of-life benefit of quiescence for life.
Once patients progress to late-stage disease, their quality-of-life will drop to the same value as recurrence, but may be increased again through successful surgery (or decreased by unsuccessful surgery) based on published utilities. The disutility for recurrence (0.035) was used for untreated late-stage DD as this is likely to reflect the utility of RIDD participants who have only just developed late-stage DD and may be Tubiana stage 2 or the top of stage 1. This was thought to be more appropriate for the RIDD sample than assuming that patients' utility goes down to the disutility of failure (0.224, based on Tubiana stage 3) as soon as they developed late-stage DD. There were insufficient patients in the RIDD trial with flexion deformity >30 to estimate utilities for late-stage DD from the trial data.
   in all other cases, the disutilities associated with each late-stage DD state were based on those used by Brazzelli et al.¹⁰ We converted Brazzelli's utilities to disutilities and subtract those disutilities from each patient's baseline utility to ensure that patients' utility did not increase when they developed late-stage DD, which would not be clinically plausible.
      Success=Tubiana Stage 0 utility 1, disutility 0
      Recurrence=Tubiana Stage 1 utility 0.965, disutility 0.035
      treatment failure=Tubiana Stage 3 utility 0.776, disutility 0.224
   During the 6-month period in which surgery occurs, patients' utility was assumed to remain at the value for the failure state; after the six-month period, patients' utility will change to the value of success or failure depending on the outcome of treatment. For simplicity, we assumed that all patients will there is no difference in the time interval between surgery and improvement in quality of life between operations in between first-line and second-line. While this is unlikely to be the case in practice 24, this simplifying assumption in late-stage DD is unlikely to have a substantial effect on the conclusions for early-stage DD.
We applied the age-related decline from Ara and Roberts Model 117 to decrease utility year-on-year as patients age. The effect of aging was applied additively, with patients' utility in cycle c(,) being equal to their pre-randomisation utility(m,), plus any modifiers capturing the effect of quiescence, late-stage DD or treatment failure, recurrence or success, plus the age effect. Following Dakin et al 2020, 29 we used the age (1) and age-squared (1) coefficients from Model 1 to estimate the impact of ageing on quality-of-life as a function of the number of years since randomisation:

$$\begin{matrix}, = , + [ ] + ( 1 \cdot + 2 \cdot ) \\ - ( 1 \cdot + 2 \cdot )\end{matrix}$$

QALYs were based on the area under the curve. Utility was estimated at the end of each cycle based on whether the patient was quiescent, had progressive early-stage DD or which late-stage DD state they were in. If the patient was alive for the whole cycle, QALYs during the six-month cycle were equal to the average of the utility at the beginning and utility at the end of the cycle, divided by two.
Successful surgery for late-stage DD was assumed to return patients to their pre-randomisation utility (minus any age-related decline in QoL). The difference between success and baseline utility was varied over a normal distribution with mean 0 and a standard error equal to that for the utilities for recurrence and failure in order to parameterize the uncertainty around this assumption.
Patients who are about to undergo their 2^{nd} or subsequent surgery for late-stage DD were assumed to have the same utility as the “failure” state, regardless of whether they have failed treatment or recurred after successful treatment. This assumption matches that of Brazzelli et al.¹⁰
For simplicity, we assumed that there was no difference in the time interval between surgery and improvement in quality of life between operations in between first-line and second-line. In practice, there is evidence that this delay is longer for more invasive surgery 24, although this simplifying assumption in late-stage DD is unlikely to have a substantial effect on the conclusions for early-stage DD.
A form of half-circle correction was applied for QALYs, in that if a patient died during that cycle, we assumed that patients died three months through the cycle and assumed a linear interpolation in utility between start of cycle and what would have been end of cycle. QALYs in the cycle patients die therefore equalled (1.5*start of cycle utility+0.5*end of cycle utility)/8.
Following Brazzelli et al,¹⁰ we applied a disutility for 6 weeks and a cost for patients who had complications relating to the hand that led to hospital intervention after late-stage DD surgery. These costs were applied in full regardless of whether the patient died in that cycle since treatment was given at the beginning of the cycle. Although Asler et al²⁵ found that primary DD surgery increases the risk of acute kidney injury, cardiovascular events and respiratory/urinary infections, we excluded these events from the model because costs unrelated to DD were excluded from the within-trial analysis and as including these outcomes could lead to double counting the excess mortality for DD, would greatly complicate the model and would have minimal impact on the results on interventions for early-stage DD. Complications and long-term adverse effects from surgery that did not lead to hospital intervention were excluded, following Brazzelli.¹⁰

ETABLE 11
Data inputs for the model-based extrapolation. Min and max represents the minimum and maximum values using one-way
sensitivity analysis to generate the tornado diagram.

| Parameter | Definition | Mean | Alpha | Beta | distribution | SE | Min | Max |
|---|---|---|---|---|---|---|---|---|
| u_Quiesence | Difference in utility between quiescent | 0.0395 | | | bootstrap | 0.0159 | 0.0079 | 0.0704 |
| | and non-quiescent patients (quiescence | | | | | | | |
| | coefficient in the equation predicting | | | | | | | |
| | the QALYs between 6 months and 18 | | | | | | | |
| | months). | | | | | | | |
| f_18m_ectopic | Coefficient for ectopic disease in the | 8.5189 | | | bootstrap | 3.2853 | 2.1683 | 15.3596 |
| | equation for change in active flexion | | | | | | | |
| | deformity between baseline and 18 | | | | | | | |
| | months. This is 3 times the change per | | | | | | | |
| | cycle | | | | | | | |
| f_placebo_cons | Constant coefficient in the equation for | 0.8631 | | | bootstrap | 1.5382 | -2.0005 | 4.1074 |
| | change in active flexion deformity | | | | | | | |
| | between baseline and 18 months. This | | | | | | | |
| | is 3 times the change per cycle | | | | | | | |
| p_late_NT | Proportion of patients in the placebo | 0.2149 | | | bootstrap | 0.0487 | 0.1286 | 0.3143 |
| | group who have late-stage DD at the | | | | | | | |
| | end of the trial | | | | | | | |
| p_late_A | Proportion of patients in the placebo | 0.1800 | | | bootstrap | 0.0494 | 0.1000 | 0.2857 |
| | group who are quiescent at the end of | | | | | | | |
| | the trial | | | | | | | |
| p_quiescent_NT | Proportion of patients in the A group | 0.2209 | | | bootstrap | 0.0556 | 0.1286 | 0.3429 |
| | who have late-stage DD at the end of | | | | | | | |
| | the trial | | | | | | | |
| p_quiescent_A | Proportion of patients in the A group | 0.3696 | | | bootstrap | 0.0614 | 0.2571 | 0.4857 |
| | who are quiescent at the end of the trial | | | | | | | |
| f_SA_Quies_A | Mlogit output - used to estimate % | 0.8023 | | | bootstrap | 0.4491 | −0.0404 | 1.7329 |
| | quiesence in sensitivity analsyis | | | | | | | |
| | controlling for flexion | | | | | | | |
| f_SA_Quies_flex | Mlogit output - used to estimate % | −0.0062 | | | bootstrap | 0.0187 | −0.0464 | 0.0279 |
| | quiesence in sensitivity analsyis | | | | | | | |
| | controlling for flexion | | | | | | | |
| f SA_Quies_cons | Mlogit output - used to estimate % | -1.1033 | | | bootstrap | 0.3999 | -1.9189 | −0.3942 |
| | quiesence in sensitivity analsyis | | | | | | | |
| | controlling for flexion | | | | | | | |
| f_SA_Late_A | Mlogit output - used to estimate % late | 0.6484 | | | bootstrap | 0.7954 | −0.8610 | 2.3087 |
| | in sensitivity analysis controlling for | | | | | | | |
| | flexion | | | | | | | |
| f_SA_Late_flex | Mlogit output - used to estimate % late | 0.1313 | | | bootstrap | 0.0308 | 0.0824 | 0.2019 |
| | in sensitivity analysis controlling for | | | | | | | |
| | flexion | | | | | | | |
| f_SA Late_cons | Mlogit output - used to estimate % late | -1.8266 | | | bootstrap | 0.6713 | -3.4881 | −0.7387 |
| | in sensitivity analysis controlling for | | | | | | | |
| | flexion | | | | | | | |
| p_suc_PNF | Probability of correction in contracture | 0.41 | 68 | 99 | beta | 0.038 | 0.335 | 0.485 |
| | to within 0-5° of full extension: PNF | | | | | | | |
| p_recurrence_PNF | 6-monthly probability of | 0.0248 | 1.6 | 56 | beta | 0.019 | −0.013 | 0.063 |
| | recurrence (i.e. return in contracture of | | | | | | | |
| | at least 20°): PNF | | | | | | | |
| p_TreatRecurrence_ | 6-month probability of further | 0.73 | 33 | 12 | beta | 0.066 | 0.601 | 0.859 |
| PNF | treatment if treatment fails/during | | | | | | | |
| | recurrence: PNF | | | | | | | |
| p_IntraOpAE_ | Probability of serious local | 0.60% | 903 | 149930 | beta | 0.000 | 0.006 | 0.006 |
| allsurgery | complications requiring further hospital | | | | | | | |
| | intervention within 90 days (excluding | | | | | | | |
| | amputation): PNF, LF & DF combined | | | | | | | |
| c_Procedure_A | Cost of adalimumab and administration | £950 | | | fixed | | 713.86 | £950 |
| | per 2 injections | | | | | | | |
| c_Procedure_PNF | Procedure cost: PNF | £1,132 | | | fixed | | £566 | £1,697 |
| c_Physio | Cost per physiotherapy session | £58 | | | fixed | | £29 | £87 |
| c_OutpatientFU | Cost per outpatient consultation | £122 | | | fixed | | £61 | £183 |
| c_Dressingchange | Cost per dressing change | £101 | | | fixed | | £0 | £152 |
| n_Physio_PNF | Number of physiotherapy appointments | 0.375 | 2.032258 | 0.184524 | gamma | 0.263 | 0.000 | 0.891 |
| | in the 6 month-period that includes | | | | | | | |
| | surgery | | | | | | | |
| n_dressingchange_ | Number of dressing changes in the 6 | 0.375 | 1 | 0.375 | gamma | 0.375 | 0.000 | 1.110 |
| PNF | month-period that includes surgery | | | | | | | |
| n_OutpatientFU_ | Number of OutpatientFU appointments | 0.375 | 0.828947 | 0.452381 | gamma | 0.412 | 0.000 | 1.182 |
| PNF | in the 6 month-period that includes | | | | | | | |
| | surgery | | | | | | | |
| c_Complications | Cost per complication requiring | £2,017 | | | fixed | | £1,009 | £3,026 |
| | treatment | | | | | | | |
| HR_DD | Hazard ratio for mortality with DD | 1.48 | | | lognormal | 0.054 | 1.000 | 1.700 |
| | compared with no DD | | | | | | | |
| u_AE | Lost QALYs from AEs: disutility of - | 0.0071 | 0.057705 | 8.074168 | beta | 0.029 | 0.000 | 0.115 |
| | 0.0615 for 6 weeks | | | | | | | |
| u_S | Disutility for the success state: i.e. how | 0 | 0 | 0 | normal | 0.020 | −0.039 | 0.039 |
| | much lower utility is for patients in the | | | | | | | |
| | success state compared with trial | | | | | | | |
| | baseline | | | | | | | |
| u_R | Disutility for the recurrence state and | 0.035 | 2.915699 | 80.39 | beta | 0.020 | −0.004 | 0.101 |
| | untreated late DD: i.e. how much lower | | | | | | | |
| | utility is for patients in the recurrence | | | | | | | |
| | state compared with trial baseline. | | | | | | | |
| u_F | Disutility for the failure, treatment and | 0.224 | 97.06763 | 336.27 | beta | 0.020 | 0.101 | 0.263 |
| | untreated late DD states: i.e. how much | | | | | | | |
| | lower utility is for patients in these | | | | | | | |
| | states compared with trial baseline | | | | | | | |
| u_age | Change in utility for each additional | −0.0002587 | | | normal | 0.000 | −0.00099 | 0.00047 |
| | year of age | | | | | | | |
| u_agesquared | Change in utility for each additional | −0.0000332 | | | normal | 0.000 | −0.00004 | −0.00003 |
| | unit of age-squared | | | | | | | |
| Duration_quiescence | Number of years that quiescent nodules | 3 | | | lognormal | 0.233752 | 1.500 | 5.000 |
| | will stay quiescent for, before starting | | | | | | | |
| | to progress | | | | | | | |
| discountrate | Discount rate for costs and QALYs | 0.035 | | | fixed | | 0.000 | 0.050 |
| p_suc_LF | Probability of correction in contracture | 0.71 | 89 | 36 | beta | 0.041 | 0.631 | 0.789 |
| | to within 0-5° of full extension: LF | | | | | | | |
| p_recurrence_LF | 6-monthly probability of | 0.0054 | 0.5 | 56 | beta | 0.008 | −0.010 | 0.020 |
| | recurrence (i.e. return in contracture of | | | | | | | |
| | at least 20°): LF | | | | | | | |
| p_TreatRecurrence_ | 6-month probability of further | 0.4 | 4 | 6 | beta | 0.155 | 0.096 | 0.704 |
| LF | treatment if treatment fails/during | | | | | | | |
| | recurrence: LF | | | | | | | |
| c_Procedure_LF | Procedure cost: LF | £1,848 | | | fixed | | £924 | £2,771 |
| n_Physio_LF | Number of physiotherapy appointments | 2.2 | 19.36 | 0.11 | gamma | 0.500 | 1.220 | 3.180 |
| | in the 6 month-period that includes | | | | | | | |
| | surgery | | | | | | | |
| n_dressingchange_ | Number of dressing changes in the 6 | 2.8 | 6.88 | 0.41 | gamma | 1.068 | 0.707 | 4.893 |
| LF | month-period that includes surgery | | | | | | | |
| n_OutpatientFU_LF | Number of Outpatient FU | 2 | 20 | 0.1 | gamma | 0.447 | 1.123 | 2.877 |
| | appointments in the 6 month-period | | | | | | | |
| | that includes surgery | | | | | | | |
| LRR_ | Estimate of the natural log of the | −0.693 | | | normal | 0.354 | -1.387 | 0.001 |
| TreatRecurrence_ | ratio between the probability of a | | | | | | | |
| DFvLF | patient who failed their last treatment | | | | | | | |
| | going on to have DF, compared with a | | | | | | | |
| | patient whose next treatment is LF | | | | | | | |
| rate_recurrence_DF | 6-monthly probability of | 0.000603 | 0.5 | 56 | beta | 0.000 | 0.001 | 0.001 |
| | recurrence (i.e. return in contracture of | | | | | | | |
| | at least 20°): DF | | | | | | | |
| c_Procedure_DF | Procedure cost: DF | £2,513 | | | fixed | | £1,256 | £3,769 |
| n_Physio_DF | Number of physiotherapy appointments | 8 | 1 | 8 | gamma | 8.000 | 0.000 | 23.680 |
| | in the 6 month-period that includes | | | | | | | |
| | surgery | | | | | | | |
| n_dressingchange_ | Number of dressing changes in the 6 | 4 | 1 | 4 | gamma | 4.000 | 0.000 | 11.840 |
| DF | month-period that includes surgery | | | | | | | |
| n_OutpatientFU_DF | Number of OutpatientFU appointments | 4 | 1 | 4 | gamma | 4.000 | 0.000 | 11.840 |
| | in the 6 month-period that includes | | | | | | | |
| | surgery | | | | | | | |

Parameters used in sensitivity analyse but not in the base case analysis

| | | | | | | | | |
|---|---|---|---|---|---|---|---|---|
| c_Procedure_RT | Cost of procedures for 6 months: | £1,910 | | | fixed | | £1,337 | £13,890 |
| | Radiotherapy | | | | | | | |
| c_Procedure_Steroid | Cost of procedures for 6 months: | £341 | | | normal | £12 | £318 | £364 |
| | steroid injections | | | | | | | |
| p_quiescent_Steroid | Proportion of patients having steroids | 0.97 | 73.00 | 2.00 | beta | 0.019 | 0.129 | 1.000 |
| | who are quiescent at the end of the 18 | | | | | | | |
| | month period | | | | | | | |
| Duration_ | Duration of quiescence with steroids | 0.50 | 0.614633 | 0.813493 | gamma | 0.638 | 0.500 | 3.000 |
| quiescence_ | | | | | | | | |
| Steroid | | | | | | | | |
| c_NonNHS_A | Non-NHS cost associated with the | £0 | | | fixed | | £0 | £0 |
| | operation (assumed to be incurred | | | | | | | |
| | straight after the procedure) | | | | | | | |
| c_NonNHS_Steroid | Non-NHS cost associated with the | £0 | | | fixed | | £0 | £0 |
| | operation (assumed to be incurred | | | | | | | |
| | straight after the procedure) | | | | | | | |
| c_NonNHS_RT | Non-NHS cost associated with the | £0 | | | fixed | | £0 | £0 |
| | operation (assumed to be incurred | | | | | | | |
| | straight after the procedure) | | | | | | | |
| c_NonNHS PNF | Non-NHS cost associated with the | £96 | £1 | £96 | gamma | £96 | £0 | £1,344 |
| | operation (assumed to be incurred | | | | | | | |
| | straight after the procedure) | | | | | | | |
| c_NonNHS_LF | Non-NHS cost associated with the | £3,552 | £1 | £3,552 | gamma | £3,552 | £1,638 | £5,760 |
| | operation (assumed to be incurred | | | | | | | |
| | straight after the procedure) | | | | | | | |
| p_suc_DF | Probability of correction in contracture | 0.71 | 89 | 36 | Linked | 0.041 | 0.631 | 0.789 |
| | to within 0-5° of full extension: DF | | | | | | | |
| p_recurrence_DF | | | | | | | | |
| p_Treat PNF | Proportion of patients who will have | | | | | | | |
| | surgery in each cycle (of those who | | | | | | | |
| | have failed their last treatment and | | | | | | | |
| | have never received PNF): If PNF is | | | | | | | |
| | next treatment | | | | | | | |
| p_Treat LF | Proportion of patients who will have | | | | | | | |
| | surgery in each cycle (of those who | | | | | | | |
| | have failed their last treatment and | | | | | | | |
| | have never received LF): If LF is next | | | | | | | |
| | treatment | | | | | | | |
| p_Treat_DF | Proportion of patients who will have | | | | | | | |
| | surgery in each cycle (of those who | | | | | | | |
| | have failed their last treatment and | | | | | | | |
| | have never received DF): If DF is next | | | | | | | |
| | treatment | | | | | | | |
| p_TreatRecurrence_ | 6-month probability of further | | | | | | | |
| DF | treatment if treatment fails/during | | | | | | | |
| | recurrence: DF | | | | | | | |
| p_IntraOpAE_DF | Probability of serious local | | | | | | | |
| | complications requiring further hospital | | | | | | | |
| | intervention (excluding amputation): | | | | | | | |
| | DF | | | | | | | |
| p_IntraOpAE_LF | Probability of serious local | | | | | | | |
| | complications requiring further hospital | | | | | | | |
| | intervention (excluding amputation): | | | | | | | |
| | LF | | | | | | | |
| p_IntraOpAE_PNF | Probability of serious local | | | | | | | |
| | complications requiring further hospital | | | | | | | |
| | intervention (excluding amputation): | | | | | | | |
| | PNF | | | | | | | |
| p_late_Steroid | probability of late-stage DD 18 months | | | | | | | |
| | after steroid treatment | | | | | | | |
| Duration_ | Duration of quiescence with | | | | | | | |
| quiescence_ | radiotherapy (years) | | | | | | | |
| RT | | | | | | | | |
| p_quiescent_RT | probability of quiescence 18 months | | | | | | | |
| | after RT treatment | | | | | | | |
| p_late_RT | probability of late-stage DD 18 months | | | | | | | |
| | after RT treatment | | | | | | | |
| c_NonNHS_DF | Non-NHS cost associated with the | | | | | | | |
| | operation (assumed to be incurred | | | | | | | |
| | straight after the procedure) | | | | | | | |

Additional fixed parameters used in base case analysis

| | | | | | | | | |
|---|---|---|---|---|---|---|---|---|
| prevalanceDD | Prevalence of DD. Used to estimate | 0.166667 | N/A | N/A | Fixed | N/A | N/A | N/A |
| | DD-specific mortality | | | | | | | |
| f_rMSE_changeflex | Root-mean squared error of the change | 12.60787 | N/A | N/A | Fixed | N/A | N/A | N/A |
| | in flexion deformity between baseline | | | | | | | |
| | and 18 months. Used to introduce | | | | | | | |
| | random variation between loops in the | | | | | | | |
| | changing flexion deformity, mirroring | | | | | | | |
| | a distribution of individual patients | | | | | | | |
| | Annual mortality rates by age and sex | | | | | | | |

| Parameter | Source |
|---|---|
| u_Quiesence | Regression on RIDD. Utilities were |
| | based on EQ-5D-5L cross-walked to |
| | EQ-5D-3L UK TTO tariff. |
| f_18m_ectopic | Regression on RIDD |
| f_placebo_cons | Regression on RIDD |
| p_late_NT | Regression on RIDD |
| p_late_A | Regression on RIDD |
| p_quiescent_NT | Regression on RIDD |
| p_quiescent_A | Regression on RIDD |
| f_SA_Quies_A | Regression on RIDD |
| f_SA_Quies_flex | Regression on RIDD |
| f SA_Quies_cons | Regression on RIDD |
| f_SA_Late_A | Regression on RIDD |
| f_SA_Late_flex | Regression on RIDD |
| f_SA Late_cons | Regression on RIDD |
| p_suc_PNF | Brazelli¹⁰ (van Rijssen et al 2012³⁰) |
| p_recurrence_PNF | Brazelli¹⁰ (van Rijssen et al 2012³⁰) |
| p_TreatRecurrence_ | van Rijssen et al 2012³⁰, following |
| PNF | Brazelli¹⁰ |
| p_IntraOpAE_ | Alser²⁵ (Table 1): incidence of serious |
| allsurgery | local complications requiring |
| | hospitalisation within 90 days of all DD |
| | surgery (all local complications, minus |
| | numbers of amputations) |
| c_Procedure_A | Cost of Humira 40 mg/0.4 ml solution for |
| | injection, 2 pre-filled syringes £704.28, |
| | plus 2 outpatient consultations (£122, |
| | weighted average of consultant-led |
| | plastic surgery and trauma & |
| | orthopaedics non-admitted face-to-face |
| | attendances), plus 2 tubes of Ametop 4% |
| | anaesthetic gel (£1.08).¹⁵ Lower limit |
| | based on Yuflyma 40 mg/0.4 ml solution |
| | containing citrate (£633.70 for 2 |
| | syringes) delivered by a GP (£39⁶). |
| | Alternative value based on the |
| | assumption that the cheapest plausible |
| | price for aconcentrated adalimumab |
| | formulation is the lowest price charged |
| | for a different anti-TNF (Erelzi |
| | 50 mg/1 ml solution of etanercept |
| | (£643.50 for 4 syringes) delivered by a |
| | GP (£39⁶)). |
| c_Procedure_PNF | Procedure code: T54.1 - Division of |
| | palmar fascia. HRG code HN45A - |
| | Minor Hand Procedures for Non- |
| | Trauma, 19 years and over,¹⁶ plus cost of |
| | splint (£37.50). Varied +−50% to |
| | generate tornado diagram. |
| c_Physio | Weighted average of all physiotherapy |
| | non-admitted face-to-face attendances, |
| | NCL (Non Consultant Led) tab.16 Varied |
| | +−50% to generate tornado diagram. |
| c_OutpatientFU | Weighted average of all first and follow- |
| | up plastic surgery and trauma & |
| | orthopaedics non-admitted face-to-face |
| | attendances, CL (Consultant Led) tab.¹⁶ |
| | Varied +−50% to generate tornado |
| | diagram. |
| c_Dressingchange | Weighted average of all follow-up |
| | plastic surgery and trauma & |
| | orthopaedics non-admitted face-to-face |
| | attendances, NCL (Non Consultant Led) |
| | tab.¹⁶ Minimum value of 0 assumes |
| | dressing changes occurred during |
| | outpatient visits rather than requiring a |
| | separate visit. Upper limit +50% of |
| | mean. |
| n_Physio_PNF | 8 patients having PNF in RIDD |
| | providing data on resource use |
| n_dressingchange_ | 8 patients having PNF in RIDD |
| PNF | providing data on resource use |
| n_OutpatientFU_ | 8 patients having PNF in RIDD |
| PNF | providing data on resource use |
| c_Complications | Based on Brazelli table 19.¹⁰ Varied +- |
| | 50% to generate tornado diagram. |
| HR_DD | Kuo et al.²⁰ Unadjusted hazard ratio for |
| | DD vs no DD in UK. 1.48; 99% CI, 1.29 |
| | to 1.70. Minimum for tornado diagram |
| | set to 1 to test the impact of assuming no |
| | excess mortality |
| u_AE | Brazelli et al.¹⁰ “this is half a decrement |
| | on the European Quality of Life-5 |
| | Dimensions-3 levels (of severity) |
| | instrument (EQ-5D-3L) of a move from |
| | no pain or discomfort to some pain or |
| | discomfort” on the UK time trade-off |
| | tariff. The QALY loss was varied |
| | between 0 and that for loss of 6 quality- |
| | adjusted weeks in tornado diagram and |
| | this range was treated as 95% CI when |
| | estimating alpha and beta. |
| u_S | Successful treatment was assumed to |
| | return patients to their baseline utility. |
| | The difference between success and |
| | baseline utility was varied over a normal |
| | distribution with a standard error equal |
| | to that for the utilities for recurrence and |
| | failure in order to parameterize the |
| | uncertainty around this assumption |
| u_R | We subtracted the utility for this state |
| | used by Brazelli¹⁰ from 1 to give a |
| | disutility; Brazelli's values were based |
| | on those from Gu study: a discrete |
| | choice experiment conducted on a UK |
| | general population sample.³¹ Estimated |
| | SE from alpha given in monograph. |
| | Maximum value is based on utility for |
| | Tubiana stage 2 |
| u_F | We subtracted the utility for this state |
| | used by Brazelli¹⁰ from 1 to give a |
| | disutility; Brazelli's values were based |
| | on those from Gu study: a discrete |
| | choice experiment conducted on a UK |
| | general population sample.³¹ Estimated |
| | SE from alpha given in monograph. |
| | Minimum value is based on Tubiana |
| | stage 2 |
| u_age | Ara & Brazier 2010, Model 1.¹⁷ |
| | Correlated with age squared, using a |
| | variance-covariance matrix supplied by |
| | the authors. This was estimated on a |
| u_agesquared | random sample of adults in England (the |
| | Health Survey for England) using the |
| | EQ-5D-3L UK time trade-off tariff. |
| Duration_quiescence | Assumption: point estimate assumes that |
| | quiescence will assumed twice as long as |
| | the trial duration as hardness and area |
| | are still decreasing at 18 months. Lower |
| | limit assumes quiescence ends at the end |
| | of the trial and the upper limit assumes it |
| | lasts just over 3 times the duration of the |
| | trial. |
| discountrate | NICE³² & HM Treasury. Varied between |
| | no discounting and 5% discout rate. |
| p_suc_LF | Brazelli¹⁰ (van Rijssen et al 2012³⁰) |
| p_recurrence_LF | Brazelli¹⁰ (van Rijssen et al 2012³⁰) |
| p_TreatRecurrence_ | van Rijssen et al 2012³⁰, following |
| LF | Brazelli¹⁰ |
| c_Procedure_LF | Procedure code: T25.2 - Digital |
| | fasciectomy. HRG: HN44A/ HN44B - |
| | Intermediate Hand Procedures for Non- |
| | Trauma, 19 years and over,¹⁶ plus cost of |
| | splint (£37.50). Varied +−50% to |
| | generate tornado diagram. |
| n_Physio_LF | 5 patients having LF in RIDD |
| n_dressingchange_ | 5 patients having LF in RIDD |
| LF | |
| n_OutpatientFU_LF | 5 patients having LF in RIDD |
| LRR_ | Expert opinion JN. SE was set such that |
| TreatRecurrence_ | the upper 95% confidence interval |
| DFvLF | indicates no difference in retreatment |
| | probability between LF and DF |
| rate_recurrence_DF | Armstrong: defining recurrence as cords |
| | that have “progressed to recontracture” |
| c_Procedure_DF | Procedure code: T56.1 - |
| | Dermofasciectomy. HRG: HN34A/ |
| | HN34B - Major Hand Procedures for |
| | Non-Trauma, 19 years and over,¹⁶ plus |
| | cost of splint (£37.50). Varied +−50% to |
| | generate tornado diagram. |
| n_Physio_DF | 1 patient having DF in RIDD. SE set to |
| | same as mean |
| n_dressingchange_ | 1 patient having DF in RIDD. SE set to |
| DF | same as mean |
| n_OutpatientFU_DF | 1 patient having DF in RIDD. SE set to |
| | same as mean |
| Parameters used in sensitivity analyse but not in the base case analysis | |
| c_Procedure_RT | Base case analysis assumes 10 doses as |
| | this is most common. Lower limit on |
| | cost represents cost for 7 doses. Point |
| | estimate and lower limit: Procedure |
| | code: X65.4 - Delivery of a fraction of |
| | external beam radiotherapy NEC; HRG |
| | code: SC97Z - Same Day Radiotherapy |
| | Admission or Attendance (excluding |
| | Brachytherapy), OPROC (Outpatient |
| | Procedure) Tab; weighted average of |
| | plastic surgery and trauma & |
| | orthopaedics.¹⁶ Upper limit based on: |
| | Procedure code: X65.4 - Delivery of a |
| | fraction of external beam radiotherapy |
| | NEC; HRG code: SC97Z - Same Day |
| | Radiotherapy Admission or Attendance |
| | (excluding Brachytherapy).¹⁶ |
| c_Procedure_Steroid | Number of injections based on Ketchum |
| | et al, counting all patients having >3 |
| | injections as though they had 3 |
| | injections, in order to get an estimate of |
| | the likely number of injections that were |
| | administered in the 1st course. Cost/dose |
| | estimated as: Hand surgery: |
| | Steroid/collagenase injection. Procedure |
| | code: S52.1 - Insertion of steroid into |
| | subcutaneous tissue |
| | HRG code: JC43A - Minor skin |
| | procedures, 19 years and over, OPROC |
| | (Outpatient Procedure) Tab; weighted |
| | average of plastic surgery and trauma & |
| | orthopaedics.¹⁶ |
| p_quiescent_Steroid | Scenario 1: Point estimate is based on |
| | the proportion of hands showing |
| | “regression of disease (ie, notable |
| | softening and flattening of the focus of |
| | disease)”.³³ Min assumes the probability |
| | of quiescence is the same as the lower |
| | 95% CI for placebo. Max based on upper |
| | 95% CI for % quiescent. The minimum |
| | and point estimate are highly optimistic |
| | because nodules were not measured |
| | objectively, data were analysed |
| | retrospectively and (unlike RIDD) did |
| | not exclude patients who did not have a |
| | history of disease progression. The study |
| | by Ketchum was used as it was the |
| | largest study on steroids identified in a |
| | systematic review.¹⁴ Scenario 2 assumes |
| | that the probability of quiescence with |
| | steroid is the same as for adalimumab |
| | (with the two varying together in PSA) |
| Duration_ | Assumption: In Ketchum, patients could |
| quiescence_ | have another course of steroids every six |
| Steroid | months. The duration of quiescence was |
| | varied between 6 months (minimum |
| | possible in the model) and three years |
| | (same as with placebo and adalimumab). |
| | Standard error was estimated assuming |
| | that the minimum and maximum |
| | represented a 95% CI |
| c_NonNHS_A | |
| c_NonNHS_Steroid | |
| c_NonNHS_RT | |
| c_NonNHS PNF | Yoon: 1 day off work¹³, valued based on |
| | weekly wage of £585/week³⁴. SE was |
| | assumed to equal the mean |
| c_NonNHS_LF | Yoon: 37 days off work¹³, valued based |
| | on weekly wage of £585/week³⁴. SE was |
| | assumed to equal the mean |
| p_suc_DF | Assume that the probability of success |
| | with DF is the same as for LF, since |
| | Bainbridge et al. found that the mean |
| | number of Tubiana stages that patients |
| | improved was very similar for LF and |
| | DF ²⁴ |
| p_recurrence_DF | Converting rate to probability |
| p_Treat PNF | Assumed to be the same as for |
| | retreatment with PNF |
| p_Treat LF | Assumed to be the same as for |
| | retreatment with LF |
| p_Treat_DF | Assumed to be the same as for |
| | retreatment with DF |
| p_TreatRecurrence_ | Equals probability for LF multiplied by |
| DF | RR for DF vs LF |
| p_IntraOpAE_DF | Assumed to be the same for all late-stage |
| | DD surgery²⁵ |
| p_IntraOpAE_LF | Assumed to be the same for all late-stage |
| | DD surgery²⁵ |
| p_IntraOpAE_PNF | Assumed to be the same for all late-stage |
| | DD surgery²⁵ |
| p_late_Steroid | Steroid scenario 1: no chance of late- |
| | stage DD within 18 months based on |
| | Ketchum. Scenario 2: Assumed to be |
| | same as for adalimumab (with the two |
| | varying together) |
| Duration_ | Assumed to be the same as for |
| quiescence_ | adalimumab and placebo |
| RT | |
| p_quiescent_RT | Assumed to be same as for adalimumab |
| p_late_RT | Assumed to be same as for adalimumab |
| c_NonNHS_DF | Assumed to be the same as for LF |
| Additional fixed parameters used in base case analysis | |
| prevalanceDD | Kuo 2020²⁰ |
| f_rMSE_changeflex | Bootstrapped from RIDD: Mean change |
| | in flexion deformity between baseline |
| | and 18 months in the placebo group (see |
| | below) |
| | Office of National Statistics National |
| | Life Tables 2017-19¹¹ |

Methods for the Analysis of within-Trial Data Used as Inputs for the Model

The multiply imputed dataset used for the within trial analysis was analysed to estimate three parameters:

Proportion of patients in each arm defined as having quiescence or late-stage DD at 18 months. Multinomial logistic regression with manual backwards stepwise regression was used to assess whether four variables that had previously been shown to influence disease progression affected the probability of being quiescent or the probability of having late-stage DD rather than continuing with progressive early-stage disease: baseline active flexion deformity; family history in a first-degree relative; ectopic disease; and age of onset in years. Quiescence and late-stage DD are defined above. We began with a model containing all variables plus treatment allocation and dropped the variable with the highest p-value for quiescence each time until the model contained only statistically significant variables (p<0.05). All models controlled for randomized treatment allocation and this was retained in the model regardless of statistical significance because this is the key driver of treatment effect. No variables had a statistically significant effect on quiescence at the 0.05 level, so the final analysis was based on the crude proportion of patients with quiescence and late-stage DD in each arm to avoid any parametric assumptions. Flexion deformity had no significant effect on quiescence (p=0.837), but significantly increased the chance of late-stage DD (p<0.001); this variable was dropped from the final model used in the base case analysis, although an mlogit model controlling for flexion deformity was used in a sensitivity analysis.

Mean change in flexion deformity between baseline and 18 months in the placebo group. This analysis was estimated on the placebo group since it is applied to patients who are not receiving active treatment and those who do not achieve quiescence. All placebo participants were included in this analysis (including those who achieve quiescence and those who progressed to late-stage DD) in order that the model can predict the rate at which untreated patients will progress to late-stage DD and allow for any subsequent periods of quiescence that may naturally occur in untreated patients. Ordinary least squares regression with manual backwards stepwise regression was used to predict the mean change in flexion deformity as a function of the same four variables: baseline active flexion deformity; family history in a first-degree relative; ectopic disease; and age of onset in years. The variable with the highest p-value was sequentially dropped until the model contained only statistically significant variables (p<0.05). In this analysis, ectopic disease was found to increase the change in flexion deformity by 8.5° (95% CI: 2.5, 14.6) over 18 months (p=0.006), while baseline flexion deformity, family history and age of onset had no significant effect. Model parameters were therefore based on ordinary least squares regression predicting flexion deformity change as a function of ectopic disease. The change in flexion deformity between baseline and 18 months was divided by 3 to obtain the mean change over each six-month period. It should be noted that changes in flexion deformity are not normally distributed.

Mean QALYs between six months and 18 months. Since no patients died during the trial, this was used as a measure of the impact that quiescence and/or treatment had on mean EQ-5D utility between six months and 18 months. QALYs accrued in the first six months of the trial were excluded, since patients had not yet received three injections. Ordinary least squares regression was used to predict QALYs as a function of baseline EQ-5D utility, treatment allocation, quiescence at 18 months and the interaction between treatment allocation and quiescence. As for other analyses, the variable with the highest p-value was sequentially dropped until the model contained only statistically significant variables (p<0.05); the interaction was dropped at the same time as either main effect. Adalimumab had no significant effect on QALYs after controlling for baseline EQ-5D and quiescence (p=0.31). Quiescence was found to increase EQ-5D utility by 0.039 (95% CI: 0.008, 0.070; p=0.014) and baseline EQ-5D was also highly significant (p<0.001). The final model therefore comprised ordinary least squares regression, controlling for quiescence at 18 months and baseline EQ-5D utility.

Given the sample size of 140 patients, it was not feasible to explore the impact of other covariates within these models. Each analysis combined results of 50 imputed datasets using Rubin's rule and was conducted in Stata version 17.

The models selected by the processes above were bootstrapped to obtain a set of correlated coefficients for use in PSA. Twenty bootstraps were independently drawn for each of the 50 imputed datasets and parameters for all of the three above analyses were estimated for each of the 1000 bootstraps. Each PSA replicate used one of the 1000 sets of coefficients. This ensured that correlations within equations and between equations were propagated into the analysis. The root mean squared error from the model predicting the mean change in flexion deformity in the placebo group was also estimated for each bootstrap; the mean of this value was used within the model as a measure of the between-patient variance.

The population of individual patient data used to run the model was based on 69 of the 70 UK patients randomized to placebo (excluding one patient who had missing data on baseline EQ-5D).

### Technical Details of the Simulation

Parameter values for use in probabilistic sensitivity analysis (PSA) were drawn from the relevant distributional using Microsoft Excel 2016. A CSV file containing the parameter values for the point estimate, minima and maxima for the tornado diagram and alternative values for many of the sensitivity analysis and probabilistic parameter values was stored and used for the simulation within Stata.

The model was built and run in Stata (StataCorp, College Station TX) version 17.

Random numbers for transitions within each loop were generated in Stata. To make sure that patients are either categorized as quiescent or late-stage or neither but never both, the random number used to assign patients to late-stage disease was set to be equal to 1 minus the random number used to assign patients to quiescence. Furthermore, any patient who has already been categorized as having quiescent disease in that loop will not be classed as having late-stage disease; this condition will only apply in extreme PSA replicates in which the probability of late-stage disease and the probability of quiescence sum to >1.

To minimize Monte Carlo error for differences between arms, patient p has the same random number for mortality in all arms of the model within cycle c of loop n; likewise for late-stage DD transitions, AE from late-stage DD interventions and flexion deformity changes. The random number will differ between patients and between loops, but not between arms. This makes sure that patients will always live for the same length of time regardless of treatment allocation and reduces the number of loops that are needed for the model to converge. However, random numbers determining whether patients are quiescent or not and whether they have late-stage DD or not will vary between arms, because the probability against which they are compared differs between arms.

For each patient who has progressive early-stage DD at the start of cycle c, flexion deformity at the end of cycle c equals their flexion deformity at the end of cycle c-1 ( . . . ), plus a value for their change in flexion deformity this cycle that is randomly sampled. The randomly sampled change in flexion deformity equals the linear prediction from the model predicting change in flexion deformity between baseline and 18 months (which depends on whether the patient has ectopic disease), plus an error term ( . . . , sampled from a normal distribution with mean of zero and a standard deviation of 12 [the root-MSE from the regression]). The error term for flexion deformity was common to all treatment groups to minimize unnecessary random differences between treatment groups, but was sampled independently in each cycle (c) in each loop (i) and for each patient (p) and each PSA replicate (b)).

$$, , , , = , , , , + ( 0 - \cdot + , , , ) / 3$$

Because the change in flexion deformity varies randomly and some patients have high flexion deformity at baseline, some patients who do not have late-stage DD at the end of the trial will have a simulated end of trial flexion deformity >30. Within the structure of the model, these patients were assigned the utilities and costs of progressive early-stage DD during the first cycle of the model and their flexion for deformity may go up or down during cycle 1; if their flexion deformity is >30 at the end of cycle 1, they will spend cycle 2 in the untreated late-stage DD state and may undergo surgery in cycle 3. By contrast, patients who have late-stage DD at the end of the trial will have untreated late-stage DD during cycle one and may have surgery in cycle 2.

Flexion deformity is updated in early-stage disease, transitions in late-stage disease and age at the end of the cycle: treatment decisions that occur in this cycle are based on the flexion deformity at the beginning of the cycle.

Within each PSA replicate in the model, the duration of quiescence was rounded to the nearest 6 months. For example, if the duration of quiescence within PSA replicate 1 was 3.2 years, patients would be retreated every 6 cycles in the adalimumab with retreatment arm and within the arms without retreatment (e.g. in cycle 4, cycle 10 & cycle 16), the quality-of-life benefits for quiescence were applied for the 1^{st} 18 months of the model and flexion deformity begins to change in cycle 7.

Monte Carlo error (MCE) was estimated as the square root of the variance³⁵ around the mean costs and QALYs in each treatment group and the incremental costs and QALYs across 100 repeated runs of the model, using the mean values for all parameters. This measure indicates the imprecision around the estimates that is introduced by simulating individual patients through random numbers and it is important to run sufficient numbers of loops of the model to estimate mean outcomes accurately and avoid over-estimating standard errors. MCE was calculated based on repeated runs of the model with point estimates because the way that the model is set up (with identical patients in each treatment group and using the same random numbers for all treatment groups within any given loop) make it difficult to reliably estimate MCE analytically.

One hundred loops were run for each of the 1000 parameter sets for each of the 69 patients in each of the treatment groups (a total of 6.9 million loops). One thousand parameter sets were used for PSA: the number needed to give SEs to ±10% accuracy.³⁶ By plotting the MCE against

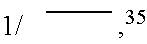

we found that 128 loops were sufficient to ensure that the MCE around the difference in modelled QALYs between adalimumab retreatment and standard care was <10% of the standard error around this measure, while 23 loops were sufficient for the MCE around the difference in cost between adalimumab once and no treatment was <10% of its SE. Since the standard error around the difference in QALYs between adalimumab once and placebo was much smaller, 334 loops would have been needed per parameter set to achieve MCE <10% of the SE, which was not feasible (estimated simulation time: 78 days) and was not deemed necessary since adalimumab once dominated no treatment. MCE was larger around total costs and total QALYs than incremental costs and QALYs because the same random numbers were used for all treatment groups (other than the transition to quiescence or late-stage DD at the end of the trial), although these outcomes are not presented for one-way sensitivity analyses or PSA.

Mean costs, QALYs and life expectancy and mean differences between groups shown in tables are based on the mean across all 1000 probabilistic parameter sets (a total of 6.9 million loops per treatment group). With 6.9 million loops, the MCE around the mean difference in modelled QALYs between adalimumab retreatment and standard care was only 0.000048. Using the mean across PSA replicates ensures that point estimates capture any nonlinearity between input parameters and outcomes. The 1000 runs of 100 loops took 23.3 computer-days to simulate using multi-user simulation servers.

It was not possible to use the formula developed by O'Hagan et al.³⁶ to eliminate bias from standard errors because the analysis has a three-level structure with loops nested in patients within each PSA replicate,³⁷ although since MCE ~10% of SEs, the degree of bias should be minimal.

However, since it was not feasible to replicate PSA for each one-way sensitivity analysis, tornado diagrams and sensitivity analyses are based on runs of the model using the mean value for each parameter shown in eTable 11, with the exception of the values changed in that sensitivity analysis. Within tornado diagrams, the base case value is based on point estimates for all parameters. Each sensitivity analysis used for the tornado diagram or scenario analysis (and the mean against which the sensitivity analyses are compared) was based on 200 loops to minimize chance differences between analyses. Chance differences between sensitivity analyses were also minimized by using the same seed for all sensitivity analyses and the mean against which the sensitivity analyses are compared. One hundred parameter sets each with 200 loops took 5 days to simulate on a multi-user server.

The model was validated by careful examination of the code, assessing the face validity of outcomes, examining the variables generated for each patient in each cycle and by running the model using a number of extreme data inputs to check that these changes had the expected result. The extreme data inputs included assuming that everyone had quiescence at the end of the trial, assuming everyone had late-stage DD at the end of the trial, applying no cost for adalimumab, applying no cost for interventions for late-stage DD, etc. Since there are very few other datasets on early DD, it was not possible to externally validate the model.

### Expected Value of Perfect Information

The expected value of perfect information (EVPI) and the expected value of perfect parameter information (EVPPI) were estimated from the 1000 probabilistic replicates using the Sheffield Accelerated Value of Information software on release version 2.2.0 (2021-06-04) of SAVI-Sheffield Accelerated Value of Information.³⁸ Results were reported at a £20,000/QALY ceiling ratio. Unless otherwise stated, EVPI and EVPPI are reported from an analysis comparing standard care, one course of adalimumab and repeated adalimumab. The seven parameters bootstrapped from the RIDD trial, plus the duration of quiescence were evaluated as a group, since all these parameters could be estimated from a new trial with longer follow-up.

The value of perfect information for the UK population over the next 10 years was based on an estimate of the number of prevalent cases of progressive early-stage DD that meet the RIDD inclusion criteria. There are very limited data on the prevalence of early-stage DD, so this figure should be treated as an estimate. The number of patients receiving treatment was not adjusted for time preference.

We applied estimates of the prevalence of different trends in Western populations at age 55, 65 and 75 from a systematic review and meta-analysis³⁹ to UK population estimates⁴⁰ (eTable 12). We assumed that the prevalence at age 55 applied to everyone in the UK population aged 50-59, that the prevalence at age 65 applied to 60-69-year-olds and that the prevalence at age 75 applied to everyone aged 70 years and over. We also conservatively assumed that there were no cases of different trends in people aged under 50 years, although it is known that this is not the case.³⁹

ETABLE 12
Number of people with Dupuytren's disease (DD) in the UK

| | Prevalence of | UK population in | No. |
|---|---|---|---|
| | Dupuyten's³⁹ | each age band⁴⁰ | cases DD |
| 55 years (assumed to | 12% | 9,126,868 | 1,095,224 |
| apply to 50-59 years) | | | |
| 65 years (assumed to | 21% | 7,211,199 | 1,514,352 |
| apply to 60-69 years) | | | |
| 75 years (assumed to | 29% | 9,153,257 | 2,654,445 |
| apply to 70+ years) | | | |
| TOTAL | | | 5,264,020 |

In the absence of UK data on the proportion of DD patients who have early-stage disease, we used data from a study in the Netherlands, which recruited a sample of patients from the general population. Of the 169 patients with DD, 81% (137) had palmar nodules without contracture (Iselin degree I) in a population-based study in the Netherlands⁴¹; on that basis, we assume that 4.27 million people in the UK have early-stage DD (81% of 5,264,020). Since 39% (112/284) patients screened for RIDD did not meet inclusion criteria,²¹ we assumed that 61% of patients with early-stage DD meet RIDD criteria. On that basis, we estimated that 2,584,411 people in the UK currently have progressive early-stage DD meeting RIDD criteria. In the absence of reliable data on incidence, we used this prevalent population as the estimate of the number of people affected by the decision over the next 10 years. There is substantial uncertainty around this figure: in particular, the proportion of people with early-stage DD may be lower in the UK than in the Netherlands and the prevalence is likely to be non-zero in under 50 s and higher than 29% in over 75's, but, conversely, many patients with progressive early-stage DD may not seek treatment or be unwilling to have injections.

### Sensitivity Analyses

The following sensitivity analyses were run to assess the uncertainty around model results:
The following analyses were done using alternative input values and results are based on 100 loops per patient:
   No excess mortality for DD. As well as testing the impact of assuming that DD does not affect patients' mortality, this tests whether conclusions are likely to be sensitive to interactions with other treatments affecting length of life.
   No discounting
   No direct utility benefit from quiescence
   No age-related decline in utility
   Adalimumab price based on Yuflyma 40 mg/0.4 ml solution containing citrate (£633.70 for 2 syringes¹⁵) delivered by a GP (£406): 715.86/6 m
   Retreatment with 3 doses of adalimumab rather than four: 712.83/6 m
   Adalimumab price £625 per 6 months (arbitrary value)
   Adalimumab price based on Yuflyma¹⁵ and 3 doses: 536.895
   Adalimumab price £470 per 6 months (arbitrary value)
   Adalimumab price based on the cheapest etanercept formulation (Erelzi 50 mg/l ml solution of etanercept (£643.50 for 4 syringes¹⁵) delivered by a GP (£406): £404.91 per 6 months)
   Using Tubiana stage 2 utilities for failure, recurrence and untreated states (utility 0.101 lower than early-stage DD¹⁰)
The following analyses were done using additional runs of the model, each with 100 loops:
   Controlling for flexion deformity when estimating probability of quiescence or late-stage DD. The base case analysis used the crude proportion of patients who were quiescent or had late-stage DD at the end of the trial as data inputs. This sensitivity analysis instead used an mlogit predicting quiescence or late-stage DD as a function of baseline flexion deformity as well as treatment allocation; this was run within the same set of bootstraps on the trial data as the base case inputs; the parameters are shown in eTable 1.
   Assuming that the sequence of late-stage DD treatments is up to three PNF procedures followed by BSC (the most cost-effective strategy evaluated by Yoon et al, which considered sequences of up to three operations), as opposed to the base case sequence (three PNF then one LF then one DF)

The following assumptions were applied in a sensitivity analysis to conduct early modelling comparing the cost-effectiveness of first-line adalimumab with first-line radiotherapy or steroids:
Radiotherapy:
   QALYs in the first 18 months with radiotherapy were assumed to be equal to the QALYs accrued in the adalimumab arm of the trial.
   The cost of radiotherapy was based on 10 doses. It was assumed that there was no difference in other costs between radiotherapy and no radiotherapy.
   This was run alongside the base case analysis and scenario 1 for steroids and is therefore based on the mean across 1000 parameter sets, each run using 100 loops.
Two scenarios were modelled for steroids:
   scenario 1 (best case):
      97% of patients were assumed to achieve quiescence, based on the proportion of early-stage DD patients who had “softening and flattening” in the study by Ketchum et al.³³
      0% of patients were assumed to progress to late-stage DD within 18 months
      This was run alongside the base case analysis for 480 of the probabilistic parameter sets, each run using 100 loops.
   Scenario 2:
      probability of quiescence and probability of late-stage DD are the same as for adalimumab
      This was run alongside the base case analysis for 587 of the probabilistic scenarios, each run using 100 loops
   In both cases:
      QALYs in the first 18 months with steroids were assumed to be equal to the QALYs accrued in the adalimumab arm of the trial.
      quiescent patients have three injections required every six months to maintain quiescence

We did not do a sensitivity analysis from a societal perspective since the within-trial analysis showed that non-NHS costs in early DD were negligible.

Subgroup analyses and analyses exploring heterogeneity were not conducted due to the size of the trial.

Additional Figures and Tables from the Model-Based Extrapolation

### Additional Results of Base Case Analysis

See eFIGS. 2-7 (FIGS. 4-9), and eTable 13, below.

ETABLE 13
Results of scenario analyses of the model-based economic evaluation with additional comparators. Values
in brackets represent 95% CI.

| | | | Standard care: | | Repeated |
|---|---|---|---|---|---|
| | | Standard care: up | up to 3 PNF, | 1 course | courses |
| | | to 3 PNF | then LF then DF | adalimumab | adalimumab |
| QALYs† | Trial | 1.279 (1.247, 1.313) | 1.279 (1.247, | 1.286 (1.248, | 1.286 (1.248, |
| | | | 1.313) | 1.320) | 1.320) |
| | Model | 9.67 (9.23, 10.04) | 10.15 (9.80, | 10.19 (9.86, | 10.45 (10.10, |
| | | | 10.49) | 10.51) | 10.79) |
| | Lifetime | 10.95 (10.49, 11.33) | 11.43 (11.08, | 11.48 (11.14, | 11.74 (11.40, |
| | | | 11.78) | 11.80) | 12.08) |
| NHS | Trial | £2,136 (£1,998, | £307 (£134, £514) | £2,136 (£1,998, | £2,697 (£2,439, |
| costs† | | £2,277) | | £2,277) | £2,888) |
| | Model | £725 (£543, £970) | £1,416 (£1,056 | £1,333 (£993, | £7,774 (£1,898, |
| | | | £1,887) | £1,780) | £9,627) |
| | Lifetime | £2,861 (£2,628, | £3,552 (£3,165, | £5,298 (£4,782, | £10,840 (£4,645, |
| | | £3,130) | £4,036) | £5,820) | £12,778) |

| Life expectancy | 22.6 (22.0, 23.2) | | 22.6 (22.0, 23.2) | 22.6 (22.0, 23.2) | 22.6 (22.0, 23.2) |
|---|---|---|---|---|---|
| Years with | 9.71 (6.39, 12.08) | | 9.71 (6.38, 12.05) | 10.39 (7.23, | 13.25 (10.52, |
| early-stage | | | | 12.72) | 15.61) |
| DD during | | | | | |
| modelled | | | | | |
| period | | | | | |
| Number of courses | 0.0 (0.0, 0.0) | | 0.0 (0.0, 0.0) | 1.0 (1.0, 1.0) | 3.65 (2.35, 5.74) |
| of treatment | | | | | |
| for early- | | | | | |
| stage DD | | | | | |
| Number of | 0.77 (0.56, 1.05) | | 1.36 (1.0, 1.88) | 1.30 (0.94, 1.79) | 0.93 (0.63, 1.32) |
| operations for | | | | | |
| late-stage DD | | | | | |
| | | | Steroids: | Steroids: | |
| | | | Scenario 1‡ | Scenario 2¶ | Radiotherapy |
| | QALYs† | Trial | 1.286 (1.248, | 1.286 (1.248, | 1.286 (1.248, |
| | | | 1.322) | 1.322) | 1.320) |
| | | Model | 11.01 (10.59, | 10.46 (10.14, | 10.19 (9.86, |
| | | | 11.46) | 10.81) | 10.51) |
| | | Lifetime | 12.30 (11.87, | 11.75 (11.42, | 11.48 (11.14, |
| | | | 12.75) | 12.10) | 11.80) |
| | NHS | Trial | £2,697 (£2,439, | £4,046 (£3,908, | £2,697 (£2,439, |
| | costs† | | £2,888) | £4,187) | £2,888) |
| | | Model | £3,884 (£1,613, | £1,333 (£996, | £7,774 (£1,898, |
| | | | £5,329) | £1,803) | £9,627) |
| | | Lifetime | £6,581 (£4,068, | £5,379 (£5,018, | £10,840 (£4,645, |
| | | | £8,138) | £5,845) | £12,778) |

| | | | |
|---|---|---|---|
| Life expectancy | 22.6 (22.0, 23.2) | 22.6 (22.0, 23.3) | 22.6 (22.0, 23.2) |
| Years with | | | |
| early-stage | 21.10 (20.39, | 13.21 (10.52, | 10.38 (7.28, 12.77) |
| DD during | 21.77) | 15.65) | |
| modelled | | | |
| period | | | |
| Number of courses | 38.90 (11.48, | 16.47 (5.73, | 1.0 (1.0, 1.0) |
| of treatment | 46.16) | 23.75) | |
| for early- | | | |
| stage DD | | | |
| Number of | 0.03 (0.0, 0.09) | 0.93 (0.63, 1.32) | 1.30 (0.94, 1.80) |
| operations for | | | |
| late-stage DD | | | |

* p <0.05
†Discounted at 3.5% per annum
‡Based on a run of probabilistic sensitivity analysis with 480 parameter sets, each with 100 loops that overlap with the base case analysis but not scenario 2. 97% of patients were assumed to achieve quiescence, based on the proportion of early-stage DD patients who had “softening and flattening” in the study by Ketchum et al.³³ 0% of patients were assumed to progress to late-stage DD within 18 months.
¶Based on a run of probabilistic sensitivity analysis with 480 parameter sets, each with 100 loops that overlap with the base case analysis but not scenario 1. Probability of quiescence and probability of late-stage DD was the same as for adalimumab.
Abbreviations: DD, Dupuytren's disease; QALY, quality-adjusted life-year

### EXAMPLE 2 REFERENCES

1. Nanchahal J, Ball C, Davidson D, et al. Anti-Tumour Necrosis Factor Therapy for Dupuytren's Disease: A Randomised Dose Response Proof of Concept Phase 2a Clinical Trial. EBioMedicine 2018; 33:282-88.
2. Png M E, Dritsaki M, Gray A, et al. Economic evaluation plan of a randomised controlled trial of intra-nodular injection of anti-TNF and placebo among patients with early Dupuytren's disease: Repurposing Anti-TNF for Treating Dupuytren's Disease (RIDD). Wellcome Open Res 2018; 3:156.
3. Adams J, Barratt P, Rombach I, et al. The clinical and cost effectiveness of splints for thumb base osteoarthritis: a randomized controlled clinical trial. Rheumatology (Oxford) 2020
4. van Hout B, Janssen M F, Feng Y S, et al. Interim scoring for the EQ-5D-5L: mapping the EQ-5D-5L to EQ-5D-3L value sets. Value Health 2012; 15:708-15.
5. Curtis L, Burns A. Unit Costs of Health and Social Care 2018. Personal Social Services Research Unit, University of Kent, Canterbury 2018
6. Curtis L, Burns Unit Costs of Health and Social Care 2020. A. doi.org/10.22024/UniKent % 2F01.02.84818 (date last accessed 29 Dec. 2020).
7. Faria R, Gomes M, Epstein D, et al. A guide to handling missing data in cost-effectiveness analysis conducted within randomised controlled trials. Pharmacoeconomics 2014; 32:1157-70.
8. White I R, Royston P, Wood A M. Multiple imputation using chained equations: Issues and guidance for practice. Stat Med 2011; 30:377-99.
9. Fenwick E, O'Brien B J, Briggs A. Cost-effectiveness acceptability curves—facts, fallacies and frequently asked questions. Health Econ 2004; 13:405-15.
10. Brazzelli M, Cruickshank M, Tassie E, et al. Collagenase clostridium histolyticum for the treatment of dupuytren's contracture: Systematic review and economic evaluation. Health Technology Assessment 2015; 19:1-201.
11. Office for National Statistics. National life tables: UK. www.ons.gov.uk/peoplepopulationandcommunity/birthsdeathsandmarriages/lifeexpectancies/datasets/nationallifetablesunitedkingdomreferencetables (date last accessed 17 Jan. 2021).
12. Chan J K-K, Werker P M N, Nanchahal J. Dupuytren's Disease. Neligan's Plastic Surgery 5th edition, Volume 6 Hand and Upper Limb: Elsevier 2022.
13. Yoon A P, Kane R L, Hutton D W, et al. Cost-effectiveness of Recurrent Dupuytren Contracture Treatment. JAMA Netw Open 2020; 3: e2019861.
14. Ball C, Izadi D, Verjee L S, et al. Systematic review of non-surgical treatments for early dupuytren's disease. BMC Musculoskelet Disord 2016; 17:345.
15. National Institute for Health and Care Excellence. British National Formulary. https://bnf.nice.org.uk/(date last accessed January 2021).
16. Department of Health. National Cost Collection: National Schedule of NHS costs-Year 2018-19-NHS trust and NHS foundation trusts. https://data.gov.uk/dataset/952bfac9-414f-4ebe-af23-357297ffc64d/health-trust-reference-costs-2018-19 (date last accessed 15 Mar. 2021).
17. Ara R, Brazier J E. Populating an economic model with health state utility values: moving toward better practice. Value Health 2010; 13:509-18.
18. National Institute for Health and Care Excellence. NICE health technology evaluations: the manual. www.nice.org.uk/process/pmg36/resources/nice-health-technology-evaluations-the-manual-pdf-72286779244741 (date last accessed 7 Feb. 2022).
19. Dritsaki M, Rivero-Arias O, Gray A, et al. What do we know about managing Dupuytren's disease cost-effectively? BMC Musculoskelet Disord 2018; 19:34.
20. Kuo R Y L, Ng M, Prieto-Alhambra D, et al. Dupuytren's Disease Predicts Increased All-Cause and Cancer-Specific Mortality: Analysis of a Large Cohort from the U.K. Clinical Practice Research Datalink. Plast Reconstr Surg 2020; 145: 574e-82e.
21. Nanchahal J, Ball C, Rombach I, et al. Anti-tumour necrosis factor therapy for early-stage Dupuytren's disease (RIDD): a phase 2b, randomised, double-blind, placebo-controlled trial. Lancet Rheumatology 2022; 4: e407-16.
22. Kadhum M, Smock E, Khan A, et al. Radiotherapy in Dupuytren's disease: a systematic review of the evidence. J Hand Surg Eur Vol 2017; 42:689-92.
23. National Institute of Health and Care Excellence (NICE). Radiation therapy for early Dupuytren's disease: Interventional procedure consultation document [IPG573]. www.nice.org.uk/guidance/ipg573 (date last accessed 9 Feb. 2021).
24. Bainbridge C, Dahlin L B, Szczypa P P, et al. Current trends in the surgical management of Dupuytren's disease in Europe: an analysis of patient charts. Eur Orthop Traumatol 2012; 3:31-41.
25. Alser O, Craig R S, Lane J C E, et al. Serious complications and risk of re-operation after Dupuytren's disease surgery: a population-based cohort study of 121,488 patients in England. Sci Rep 2020; 10:16520.
26. Crean S M, Gerber R A, Le Graverand M P, et al. The efficacy and safety of fasciectomy and fasciotomy for Dupuytren's contracture in European patients: a structured review of published studies. J Hand Surg Eur Vol 2011; 36:396-407.
27. Mendelaar N H A, Poelstra R, van Nieuwenhoven C A, et al. Outcome of Recurrent Surgery in Dupuytren's Disease: Comparison with Initial Treatment. Plast Reconstr Surg 2019; 144: 828e-35e.
28. Pharoah P D, Hollingworth W. Cost effectiveness of lowering cholesterol concentration with statins in patients with and without pre-existing coronary heart disease: life table method applied to health authority population. BMJ 1996; 312:1443-8.
29. Dakin H, Eibich P, Beard D, et al. The use of patient-reported outcome measures to guide referral for hip and knee arthroplasty. Bone Joint J 2020; 102-B: 950-58.
30. van Rijssen A L, Ter Linden H, Werker P M N. Five-year results of a randomized clinical trial on treatment in Dupuytren's disease: percutaneous needle fasciotomy versus limited fasciectomy. Plast Reconstr Surg 2012; 129:469-77.
31. Gu N Y, Botteman M F, Gerber R A, et al. Eliciting health state utilities for Dupuytren's contracture using a discrete choice experiment. Acta Orthopaedica 2013; 84:571-8.
32. National Institute for Health and Care Excellence. Guide to the methods of technology appraisal 2013. http://publications.nice.org.uk/pmg9 (date last accessed 6 Apr. 2016).
33. Ketchum L D, Donahue T K. The injection of nodules of Dupuytren's disease with triamcinolone acetonide. J Hand Surg Am 2000; 25:1157-62.
34. Office for National Statistics. Employee earnings in the UK: 2019. www.ons.gov.uk/employmentandlabourmarket/peopleinwork/earningsandworkinghours/bulle tins/annualsurveyofhoursandearnings/2019 (date last accessed 4 Feb. 2022).
35. Koehler E, Brown E, Haneuse S J. On the Assessment of Monte Carlo Error in Simulation-Based Statistical Analyses. Am Stat 2009; 63:155-62.
36. O'Hagan A, Stevenson M, Madan J. Monte Carlo probabilistic sensitivity analysis for patient level simulation models: efficient estimation of mean and variance using ANOVA. Health Econ 2007; 16:1009-23.
37. Dakin H A, Leal J, Briggs A, et al. Accurately Reflecting Uncertainty When Using Patient-Level Simulation Models to Extrapolate Clinical Trial Data. Med Decis Making 2020; 40:460-73.
38. Strong M, Oakley J E, Brennan A. Estimating multiparameter partial expected value of perfect information from a probabilistic sensitivity analysis sample: a nonparametric regression approach. Med Decis Making 2014; 34:311-26.
39. Lanting R, Broekstra D C, Werker P M N, et al. A systematic review and meta-analysis on the prevalence of Dupuytren disease in the general population of Western countries. Plast Reconstr Surg 2014; 133:593-603.
40. Office for National Statistics. Population estimates for the UK, England and Wales, Scotland and Northern Ireland: mid-2020. www.ons.gov.uk/peoplepopulationandcommunity/populationandmigration/populationestimat es/bulletins/annualmidyearpopulationestimates/mid2020 #age-structure-of-the-uk-population (date last accessed 4 Feb. 2022).
41. Lanting R, van den Heuvel E R, Westerink B, et al. Prevalence of Dupuytren disease in The Netherlands. Plastic and reconstructive surgery 2013; 132:394-403.

What is claimed is:

1. A method for maintaining quiescence in an early stage Dupuytren's disease nodule on the hand of an individual who has achieved quiescence in such nodule after receiving four initial injections of a liquid pharmaceutical composition containing 40 mg of adalimumab into such nodule, which comprises
injecting the same pharmaceutical composition into the same nodule four times at a time interval of three months between each injection over a time period of 12 months commencing at least 18 months after the fourth of the initial four injections,
wherein
(A) quiescence in such nodule is defined as meeting the following three criteria 18 months after the first of the initial four injections:
(1) decrease or no change in nodule area,
(2) decrease or no increase in nodule hardness, and
(3) less than or equal to a 5 degree increase in active flexion deformity,
(B) the initial four injections are administered at a time interval of three months between each injection over a time period of 12 months using a syringe with a 25-gauge needle, and
(C) the pharmaceutical composition is citrate-free and has a volume of 0.4 ml.

2. The method of claim 1, wherein the injecting commences 18 to 24 months after the fourth of the initial four injections.

3. The method of claim 1, wherein the injecting commences 24 to 36 months after the fourth of the initial four injections.

4. The method of claim 1, wherein the injecting commences 36 to 48 months after the fourth of the initial four injections.

5. The method of claim 1, wherein the injecting commences two to five years after the fourth of the initial four injections.

6. The method of claim 1, wherein the injecting commences five to ten years after the fourth of the initial four injections.
